(19) United States
(12) Patent Application Publication
(10) Pub. No.: US 2026/0315117 A1
(43) Pub. Date: Oct. 15, 2026

(54) PATIENT-SPECIFIC GUIDES AND IMPLANTS WITH BREAKAWAY FEATURES

(71) Applicant: Carlsmed, Inc., Carlsbad, CA (US)

(72) Inventors: Niall Patrick CASEY, Carlsbad, CA (US); Sarah JAUREGUI, San Diego, CA (US); Jeremy WINSTON, San Diego, CA (US)

(21) Appl. No.: 19/554,475

(22) Filed: Mar. 2, 2026

Related U.S. Application Data
(60) Provisional application No. 63/766,343, filed on Mar. 3, 2025.

(51) Int. Cl. A61B 17/70; A61B 17/00; A61B 17/17; A61B 17/86; A61F 2/30; A61F 2/44; A61F 2/46

(52) U.S. Cl. CPC A61B 17/7059; A61B 17/1728; A61B 17/8605; A61F 2/30749; A61F 2/30942; A61F 2/4455; A61F 2/46; A61B 2017/00477; A61F 2002/30622; A61F 2002/4687

(57) ABSTRACT

The present technology provides a patient-specific implant including a fixation plate configured to mate with anatomical elements and a plurality of guide elements extending away from the fixation plate at different orientations and each being connected to the plate by a respective breakaway connection such that a passageway of the guide element is aligned with a corresponding one of the openings in the plate to allow a drill bit, an anchor, or another item to pass along the passageway, through the corresponding one of the openings, and into one of the plurality of vertebrae. The breakaway connection is configured to break apart at a location within a recessed region.

### CROSS-REFERENCE TO RELATED APPLICATION

This application claims priority to U.S. Provisional Application No. 63/766,343, filed Mar. 3, 2025, which is hereby incorporated by reference in its entirety.

### TECHNICAL FIELD

The present disclosure is related generally to orthopedic devices with removable features, and more particularly to guides and implants with breakaway features.

### BACKGROUND

Orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper-or hypo-), and irregular spinal displacement (e.g., spondylolisthesis). Other spinal disorders that can be treated using orthopedic implants include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Non-limiting and non-exhaustive descriptions are described with reference to the following drawings. The components in the figures are not necessarily to scale, emphasis instead being placed upon illustrating principles.

FIG. 1 is an isometric view of a patient-specific implant system positioned along a patient's spinal column in accordance with embodiments of the present technology.

FIG. 2 is an isometric view of the patient-specific implant system of FIG. 1 after a fixation plate assembly has been anchored to the patient's spinal column in accordance with embodiments of the present technology.

FIG. 3 is an isometric view of a fixation plate assembly in accordance with embodiments of the present technology.

FIG. 4 is a side view of the fixation plate assembly of FIG. 3 in accordance with embodiments of the present technology.

FIG. 5 is a front view of the fixation plate assembly of FIG. 3 in accordance with embodiments of the present technology.

FIG. 6 is a detailed cross-sectional view of the fixation plate assembly taken along line 6 -6 of FIG. 3 in accordance with embodiments of the present technology.

FIG. 7 is a detailed view of a portion of the fixation plate assembly of FIG. 6.

FIG. 8 is a flowchart of a method for implanting a patient-specific implant at a target position in accordance with embodiments of the present technology.

FIGS. 9-14 show steps of a method for implanting a patient-specific implant in accordance with embodiments of the present technology.

FIG. 15 is an isometric view of a breakaway guide element of a fixation plate assembly in accordance with embodiments of the present technology.

FIG. 16 is a cross-sectional view of the portion of the patient-specific implant of FIG. 15 showing breakaway guide elements.

FIG. 17 is a cross-sectional view of the portion of the patient-specific implant after the guide element has been separated from the plate.

FIG. 18 is a cross-sectional view of a portion of a fixation plate assembly in accordance with embodiments of the present technology.

FIG. 19 is a cross-sectional view of a portion of a fixation plate assembly in accordance with embodiments of the present technology.

FIGS. 20-22 are cross-sectional views of portions of patient-specific implants with fixation plates and anchors in accordance with embodiments of the present technology.

FIG. 23 is a network connection diagram illustrating a computing system for providing patient-specific medical care and configured in accordance with embodiments of the present technology.

FIG. 24 illustrates a computing device suitable for use in connection with the system of FIG. 23, in accordance with embodiments of the present technology.

FIG. 25 illustrates a surgery manager system user interface for selecting treatments, designing implants, or managing plans, in accordance with embodiments of the present technology.

FIG. 26 illustrates an implant designer graphical user interface, in accordance with embodiments of the present technology.

FIG. 27 illustrates an implant system designer graphical user interface, in accordance with embodiments of the present technology.

FIG. 28A is an isometric view of a representative patient-specific spinal fusion device configured in accordance with embodiments of the present technology.

FIG. 28B is a side view of the portion of the device of FIG. 28A in accordance with embodiments of the present technology.

FIG. 29 is an isometric view of the device of FIG. 28A.

FIG. 30 is a side view of a device implanted in the patient's spine in accordance with embodiments of the present technology.

FIG. 31 illustrates a fixation system that has been placed in the lumbar spine in accordance with embodiments of the present technology.

FIG. 32 illustrates the fixation system in accordance with embodiments of the present technology.

FIG. 33 illustrates a cross-sectional view of an implant device of FIG. 32 in accordance with embodiments of the present technology.

FIG. 34 illustrates a cross-sectional view of an implant device of FIG. 32 in accordance with embodiments of the present technology.

FIG. 35 is a flowchart of a method for designing guides for a patient-specific fixation plate in accordance with embodiments of the present technology.

FIG. 36 is a flowchart of a method for designing guides for a patient-specific fixation plate in accordance with embodiments of the present technology.

FIG. 37 is an isometric view of a patient-specific spinal fusion device having a removable guide in accordance with embodiments of the present technology.

FIG. 38 is a side view of the patient-specific spinal fusion device and multiple guides in accordance with embodiments of the present technology.

FIG. 39 is a plan view of a surgical kit in accordance with embodiments of the present technology.

### DETAILED DESCRIPTION

### I. Overview of Technology

The present technology is directed to patient-specific medical devices designed based on a patient's anatomy. For example, in many of the embodiments disclosed herein, the present technology provides patient-specific implants or guides designed to position instruments or tools during surgical procedures. Example patient-specific guides can include plates with breakaway guide elements configured to be intraoperatively removed from the plates. The plates can be anchored to the patient's body.

The patient-specific implants or guides can be orthopedic plates having integrated, breakaway drill or anchor (e.g., screw, spikes, etc.) guides that provide the correct start point, trajectory, and/or depth for drilling or fixating into a vertebral body. Screws can be passed through the plate and into pre-formed drill holes in vertebral bodies. These features can be applied to orthopedic plates used throughout the body, including, but not limited to, lumbar plates, thoracic plates, cervical plates, long bone plates, maxio-facial plates, cranial plates, lateral plates, medial plates, anterior plates, posterior plates, fracture plates, and general orthopedic plates for use with other procedures. For example, a cervical plate can include integrated, breakaway drill guides that provide a target trajectory and/or depth for drilling into an anterior face of one or more vertebral bodies to form drill holes. Fixation elements (e.g., bone screws, fixation screws, etc.) that pass through apertures of the plate.

The patient-specific devices can include breakaway features configured to enable detachment from another component. In some embodiments, the breakaway features include one or more breakaway cleaves, frangible portions, or preferentially weakened portions positioned below the surface of the plate (e.g., sub-flush). Following removal of the breakaway component (e.g., tubular drill guide), the remaining or residual features of the removed guide are configured to not irritate tissue adjacent to the tissue-contacting surface of the plate. For example, the breakaway component can be a tubular drill guide and the remaining features can be recessed tabs, torn material, or the like.

The patient-specific devices can be guides configured to provide the proper start point, trajectory, and depth for screws used to affix the plate to the spine. The guides are configured to provide clearance for screws that are co-located within the same vertebral body. The guides can be configured to sit within the screw hole so that when the guides are removed and a screw is inserted, the screw head covers the remaining breakaway features. The breakaway tabs can be configured to mate with features on the screw to provide resistance to screw migration or back-out. The remaining tabs can be angled to provide resistance to unthreading. The drill guide can be configured to allow spikes to be used as a fixation means (e.g., plate to anatomy).

In some embodiments, the present technology provides a patient-specific implant including a fixation plate configured to mate with anatomical elements and a plurality of a guide elements extending away from the fixation plate at different orientations and each being connected to the plate by a respective breakaway connection such that a passageway of the guide element is aligned with a corresponding one of the openings in the plate. A drill bit, anchor, or another item can pass along the passageway, through the corresponding one of the openings, and into one of the plurality of vertebrae. The breakaway connections are configured to break apart at sub-flesh locations, such as within the recessed regions of the plate.

Without being bound by theory, intervertebral implants provide the most efficacy when they are implanted at the correct position. This is especially true for “patient-specific” intervertebral implants, which, as described in detail below, are designed to mate with specific anatomical targets. However, depending on the surgical approach and the type of device being implanted, it can be difficult to deliver intervertebral implants (e.g., plates, interbody devices such as cages, screw and rod systems) to precise target locations in an intervertebral disc space. For example, because the disc space is between two vertebral bodies, the precise target location often cannot be directly seen and/or accessed by the surgeon performing the implant procedure. The target location is also typically at the bottom of a narrow surgical corridor that can further reduce the ability of a surgeon to see the target site and/or reduce maneuverability of the implant once proximate to the target site. The present technology thus provides systems, devices, and associated methods that direct the intervertebral implants into the target position without having to directly visualize the target size. Without being bound by theory, the present technology is therefore expected to improve the accuracy with which interbody devices can be delivered to relatively “difficult to visualize” target positions, such as intervertebral disc spaces.

The implants described herein can therefore have at least two functions: ensuring the interbody device is positioned at the correct (and relatively difficult-to-visualize) target position and reducing movement of the interbody device once it is located at the target position. Plates can be designed to mate with a portion of patient anatomy at a second target position that is relatively easier to visualize and/or access than the interbody target position. For example, the plates can be designed to mate with one or more surfaces of the vertebral body (e.g., an anterior surface of a vertebral body, a lateral surface of a vertebral body, etc.). The plates can also be coupled to the interbody device to form a predefined three-dimensional spatial relationship therebetween. For example, when the plate is coupled to the interbody device, the interbody device can have a predefined position, orientation, alignment, and/or geometry relative to the plate. The predefined three-dimensional orientation can thus be designed to ensure that, when the plate is positioned at the relatively easier to visualize plate target position, the interbody device is directed into the relatively harder to visualize interbody target position. Thus, the plate can be used to help guide the interbody device into position. The plate can be mechanically coupled to the spine by one or more fasteners (e.g., bone screws, anchors, etc.). The fasteners can further prevent or limit movement of the plate relative to the adjacent vertebrae.

In some embodiments, a patient-specific intervertebral implant includes an intervertebral cage with guides configured to receive, for example, drills, screws, anchors, etc. The intervertebral cage can include a main body configured to mate with endplates of adjacent vertebral bodies, a patient-specific positioning feature configured to mate with a target region of at least one of the adjacent vertebral bodies. After drilling holes in anatomical element(s) using the guides, the guides can be removed from the intervertebral cage. One or more bone screws can then be delivered into the drill holes in the anatomical element(s), thereby anchoring the main body to the intervertebral cage anatomical element(s). The anatomical element(s) can include vertebral bodies, facets, etc.

Plates can be coupled to intervertebral cages by one or more connection mechanisms. The connection mechanisms can couple or be configured to couple the patient-specific positioning feature to the intervertebral cage, to maintain a predetermined configuration of the patient-specific intervertebral implant, when the intervertebral cage is between the endplates and the patient-specific positioning feature contacts the target region. The predetermined configuration can correspond to a target spatial relationship between the cage and positioning feature. Connection mechanisms can be breakaway features, pins, threaded members (e.g., screws, bolts, etc.), fasteners, breakaway features, or the like. In some embodiments, an intervertebral cage assembly can include an intervertebral cage and a removable guide detachably coupled to the cage by, for example one or more threaded members. After implantation, a physician can rotate the threaded members to decouple the guide from the intervertebral cage. The guide can then be removed from the patient. In some procedures, multiple guides can be temporarily coupled to the intervertebral cage to deliver various components for anchoring. For example, a first guide can be used to guide a drill bit for drilling. The guide can then be replaced with a bone screw guide for assisting with delivery of bone screws. A surgical kit can include multiple implants, guides, and instruments.

The present technology thus provides systems and methods for designing and implanting “patient-specific” or “personalized” medical devices that are expected to mitigate at least some of the disadvantages of conventional implants. In particular, the present technology provides systems and methods for designing and implanting patient-specific implants that are optimized for the patient's particular characteristics (e.g., condition, anatomy, pathology, medical history, etc.). For example, the patient-specific medical device can be designed and manufactured specifically for the particular patient, rather than being an off-the-shelf device. However, it shall be appreciated that a patient-specific or personalized medical device can include one or more components that are non-patient-specific, and/or can be used with an instrument or tool that is non-patient-specific. For example, patient-specific positioning features can be used with non-patient-specific articulating intervertebral implants, fixed intervertebral implants, cages, etc. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof. The implants can include positioning features selected based on the implantation site, delivery paths, or the like. Positioning features can be or include, for example, plates, plate assemblies (e.g., plates and one or more fasteners, plates with locators, etc.), arms (e.g., deployable or nondeployed arms), or combinations thereof.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise. Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

### II. Patient-Specific Vertebral Implants

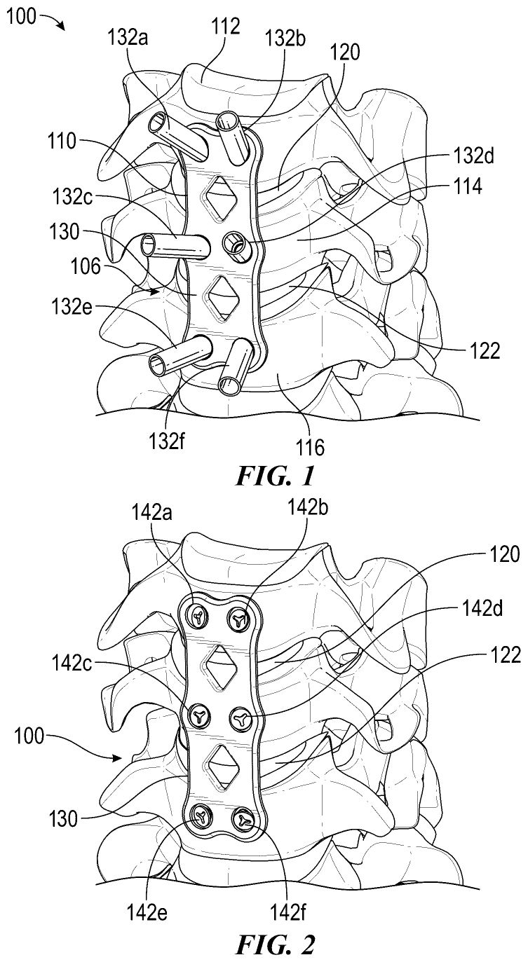

FIG. 1 illustrates a patient-specific implant system 100 positioned along a human patient's spine 106. The patient-specific implant system 100 includes a fixation plate assembly 110 positioned along vertebrae 112, 114, 116 and implants 120, 122 implanted in intervertebral disc spaces. The implant 120 is implanted between vertebrae 112, 114, and the implant 122 is implanted between vertebrae 114, 116. The fixation plate assembly 110 includes a fixation plate 130 and removable guide element 132a, 132b, 132c, 132d, 132e, 132f (collectively “guide elements 132”). The guide elements 132 can be in a patient-specific arrangement to guide, for example, needles (e.g., biopsy needles), drill bits, anchors, screws, etc.

The guide elements 132 can define, for example, target insertion locations, trajectories, depths, or the like for installing anchors to affix the plate to the spine. The guide elements 132 can be configured to allow spikes to be used as temporary or permanent fixation means (e.g., temporary plate to anatomy). For example, the guide elements 132 can be configured to provide clearance for anchors that are co-located within the same vertebral body. The guide elements 132 can sit within a recess or screw hole of the fixation plate 130 so that when the guide elements 132 are removed and a screw is inserted, a screw head of the screw can partially or completely cover the remaining breakaway features. An optional anti-back-out locking cam can keep the screw in place by partially blocking movement of the screw after the screw has been appropriately delivered. In some embodiments, the residual portions of breakaway features can be configured to mate with features of the screw to provide resistance to, for example, screw migration, screw back-out, or the like.

FIG. 2 illustrates the patient-specific implant system 100 after the guide elements 132 have been removed from the fixation plate 130. Referring to FIGS. 1 and 2, the guide elements 132 can extend away from the fixation plate 130 at different orientations to define trajectories for access target locations or regions of vertebrae. The guide elements 132 can be connected to the plate by breakaway connections configured to break apart at a location that will not contact tissue. Heads of anchors 142 can cover residual portions of the breakaway connections connected to the fixation plate 130 because the residual portions may be unsuitable for contacting tissue. The implanted fixation plate 130 can inhibit, limit, or substantially prevent relative movement between the vertebrae 112, 114, 116 to, for example, stabilize the spine, facilitate fusion and bone healing, inhibit movement of implants 120, 122, or the like.

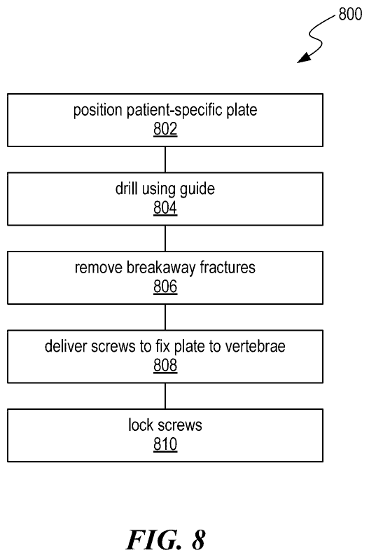

In some procedures, one or both implants 120, 122 are patient-specific implants designed to achieve a corrected anatomical configuration. The implanted fixation plate 130 can be designed based on the corrected anatomical configuration. Anchors 142a, 142b, 142c, 142d, 142e, 142f (collectively “anchors 142”) extend through the fixation plate 130 to anchor fixation plate 130 to vertebrae 112, 114, 116, thereby stabilizing the spine 106 at the corrected anatomical configuration. Implants (or implant features) can be individual or collectively designed, concurrently designed, sequentially designed, or combinations thereof. For example, a set of intervertebral cages can be concurrently designed to achieve an anatomical correction. After designing the intervertebral cages, a set of fixation plates can be designed to hold anatomical features in the corrected configuration. In some procedures, a posterior fixation system and a set of intervertebral cages can be concurrently designed to achieve an anatomical correction. Ancillary components (e.g., a set of fixation plates, a set of screws, a set of anchors, etc.) can be designed for installing the posterior fixation system, cages, etc. Example anatomical corrections, implantation steps and procedures are discussed in connection with FIGS. 8-22.

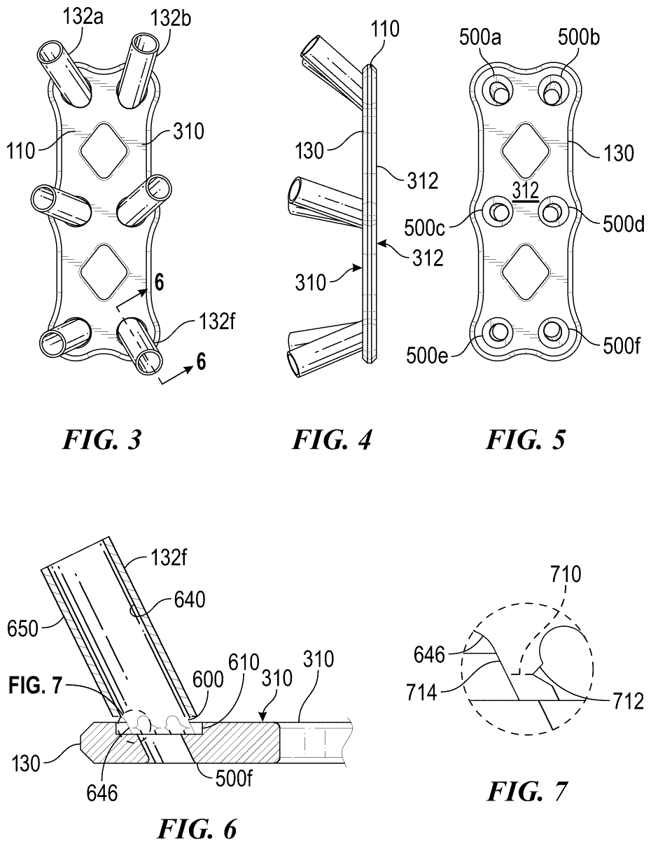

FIG. 3 is an isometric view of the fixation plate assembly 110 in accordance with embodiments of the present technology. FIG. 4 is a side view of the fixation plate assembly 110 of FIG. 3. FIG. 5 is a front view of the fixation plate assembly 110 of FIG. 3. Referring now to FIG. 3, the fixation plate 130 can be configured to seat against anatomical elements and can have a rear face 310 and a front face 312 (FIG. 3). The rear face 310 can have a generally smooth surface for atraumatically interacting with tissue. The front face 312 (FIG. 4) can be contoured to, for example, mate with, seat against, receive, or otherwise interact with anatomical elements. For example, the front face 312 can include one or more recessed regions, contoured regions, protrusions, or other features matching contours of anatomical elements, being received between anatomical elements, etc. such that the front face 312 inhibits, limits, or prevent movement of the fixation plate 130 relative to the patient's anatomy. In some embodiments, the front face 312 can include one or more transverse recessed or concave regions designed to match the sidewalls of vertebral bodies. Optionally, the front face 312 can include one or more protrusions, fixation elements, and/or features for limiting or preventing movement of the fixation plate 130 relative to a vertebral disc space. For example, a protrusion can extend from the front face 312 and into a disc space and, in some embodiments, can be coupled to an intervertebral cage (e.g., implants 120 and/or 122 in FIGS. 1 and 2) in the disc space, vertebral endplates, or the like.

The configuration, thickness, and mechanical characteristics of the fixation plate 130 can be selected based on the surgical procedure. In anterior fixation procedures, the fixation plate 130 can be configured to seat against anterior regions of vertebral bodies. In transforaminal lumbar interbody fusion (TLIF) procedures, the fixation plate 130 can be configured to be positioned along or adjacent to the facet joints. Additionally, the dimensions and size of the fixation plate 130 can be selected based on the implantation location, such as the cervical region, lumbar region, or other regions along a spine. For example, a cervical plate 130 can have a length less than a lumber plate 130 designed to fuse together the same number of vertebrae. In anterior lumbar interbody fusion (ALIF) procedures, the fixation plate 130 can be configured to be positioned along anterior regions of vertebral bodies. The fixation plate 130 can also be designed for posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF).

Referring now to FIGS. 3 and 4, the description of one guide element 132 applies to the other guide elements 132 unless indicated otherwise. The guide elements 132 can have configurations for receiving the same or different items. In some embodiments, the guide elements 132 are used for positioning drill bits. Advantageously, the guide elements 132 can define trajectories for the drill bits for drilling holes in suitable regions of vertebrae for anchoring. In some procedures, the guide element 132a is configured to receive a drill bit having a diameter smaller than a diameter of a drill bit receivable by the guide element 132b. For example, a ratio of the diameters of the passageways of the guide elements 132a, 132b can be, for example, 0.9, 0.8, 0.7, 0.6, 0.5, or ranges encompassing such ratios. This allows slender drills to be delivered through the guide element 132a. The ratios of diameters of pairs of guide elements 132 can be selected based on the anchors to be installed. The size and configuration of the guide elements 132 can be selected based on size and configuration of drill bits, anchors to be installed, characteristics of the vertebrae, and/or other parameters for the procedure.

Referring to FIG. 5, the fixation plate 130 can have a plurality of openings 500a, 500b, 500c, 500d, 500e, 500f (collectively “openings 500”). Each of the openings 500 can be configured to receive an externally threaded body of an anchor. A diameter of the openings 500 can be different based on the configuration of the anchor to be used. For example, a diameter of the opening 500c can be smaller than the diameter of the opening 500e. This allows relatively slender bone screws to be installed into the opening 500c. The number of openings 500, pattern of openings 500, and configurations of openings 500 can be selected based on the procedure to be performed.

FIG. 6 is a cross-sectional view of the fixation plate assembly 110 taken along line 6-6 of FIG. 3. The guide element 132f can be aligned with the opening 500f (illustrated as a through hole) to allow a drill bit to pass along a passageway 640 of the guide element 132f, through the opening 500f, and into a vertebra adjacent to an exit of the opening 500f. The guide element 132f can be connected to the fixation plate 130 by a breakaway connection 600. The fixation plate 130 can have a lowered or recessed region 610 containing at least a portion of the breakaway connection 600. The breakaway connection 600 is configured to break apart at a location within the recessed region 610. For example, when the guide element 132f is separated from the fixation plate 130, the remaining portions of the breakaway connection 600 (e.g., connected to the fixation plate 130) can be lower than the rear face 310 of the fixation plate 130. This allows the remaining portions of the breakaway connection 600 to be at sub-flush positions to avoid irritating tissue overlying the rear face 310.

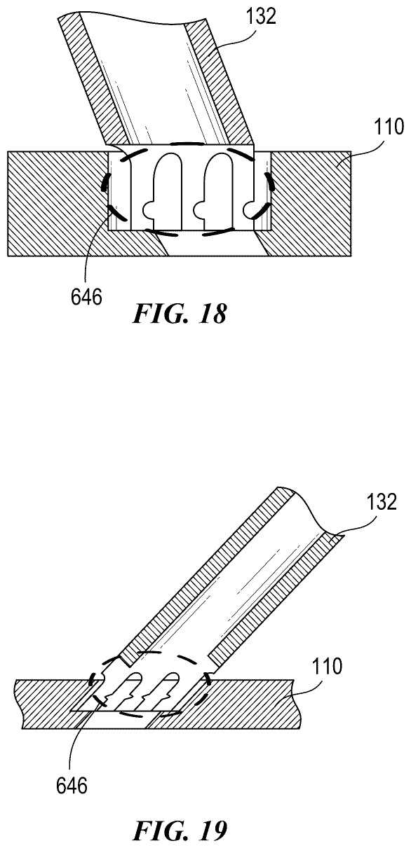

The breakaway connection 600 can include a plurality of frangible portions 646 extending from a tubular body 650 of the guide element 132f and the fixation plate 130. The frangible portions 646 (one identified in FIGS. 6 and 7) can extend to the bottom of the recessed region 610. When a user applies a force (e.g., a pulling force), torque, moment, etc. to the guide element 132f, the frangible portions 646 can break, fracture, separate, or the like. The frangible portions 646 can include, without limitation, one or more cleaves, notches, weakened portions, or the like. In some embodiments, the frangible portions 646 can extend at an angle to a lower portion of the recessed region 610. Referring to FIG. 7, the frangible portion 646 can break along a fracture line 710 extending from a notch 712 to an opposing side 714 of the frangible portion 646. The notch 712 can be a V-shaped notch (see FIGS. 17 and 19), U-shaped notch (see FIG. 18), or the like. For example, a V-shaped notch can be configured to promote crack initiation and propagation when a user pulls the guide element 132f.

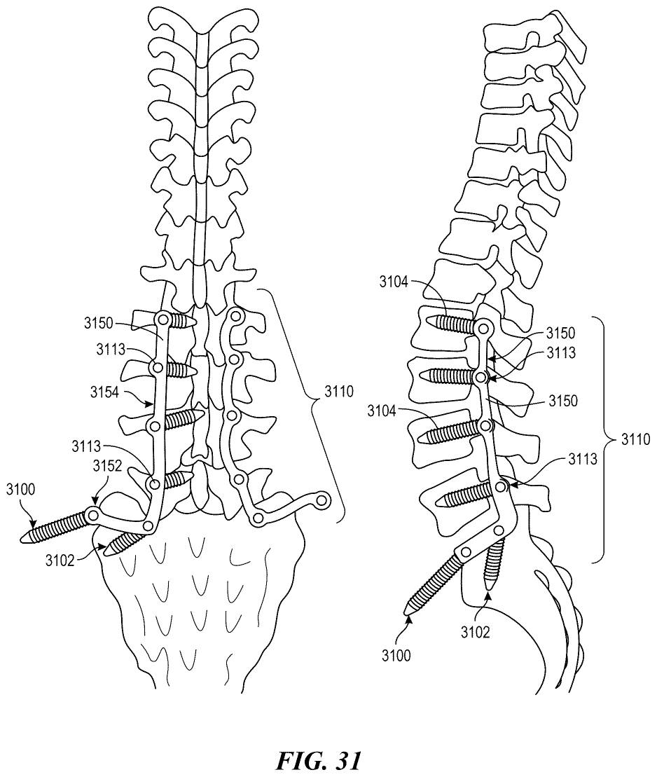

As described in connection with the system of FIGS. 31-35, the anatomy of the patient can be analyzed to design the implants of FIGS. 1-7. For example, anatomy can be analyzed to determine the configuration of frangible portions, number of breakaway features, number of anchors, trajectories of anchors, depth of anchors, and predicted post-operative outcomes. This allows the number of guides, position of guides, trajectories of guides, configurations of breakaway connections, and other features of the fixation plate assembly 110 to be selected for the patient's anatomy, planned correction, etc.

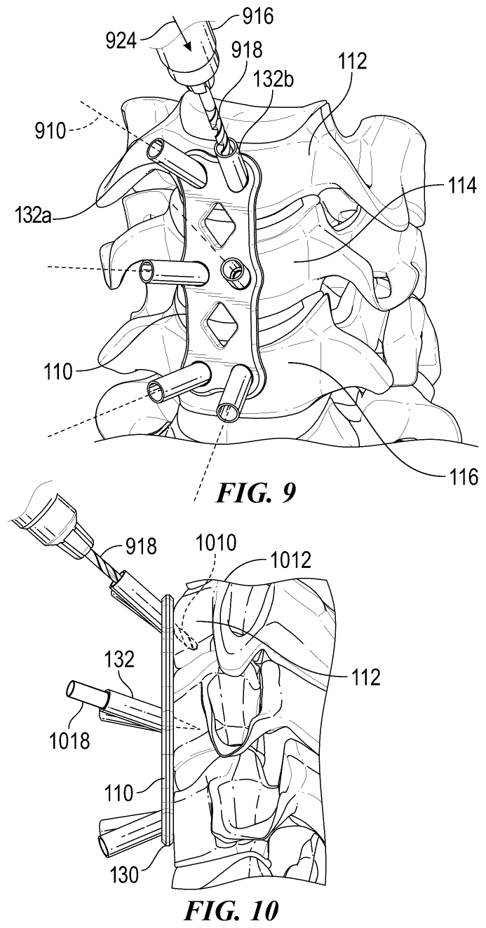

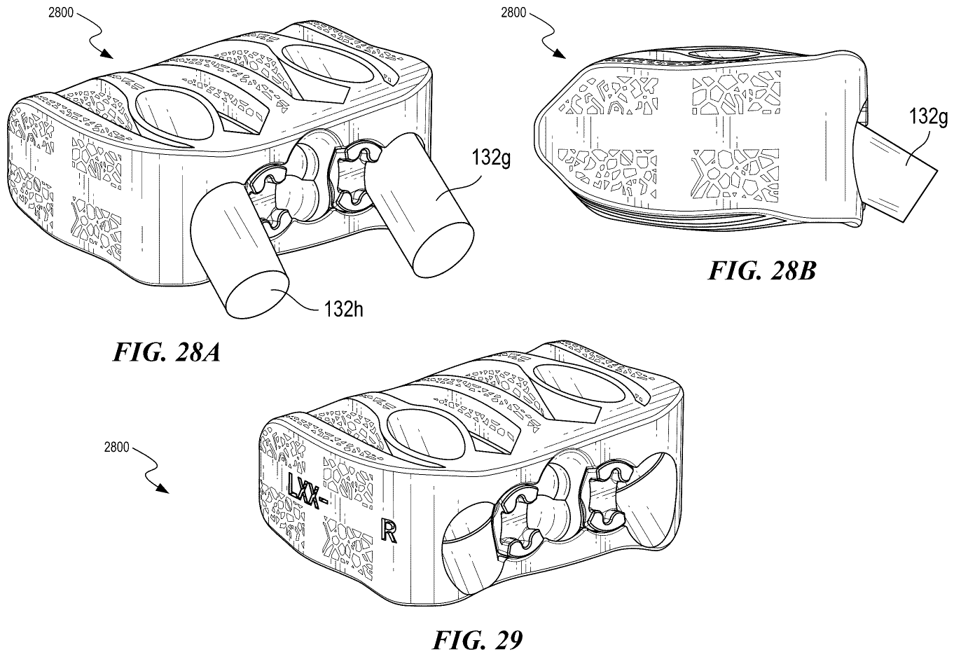

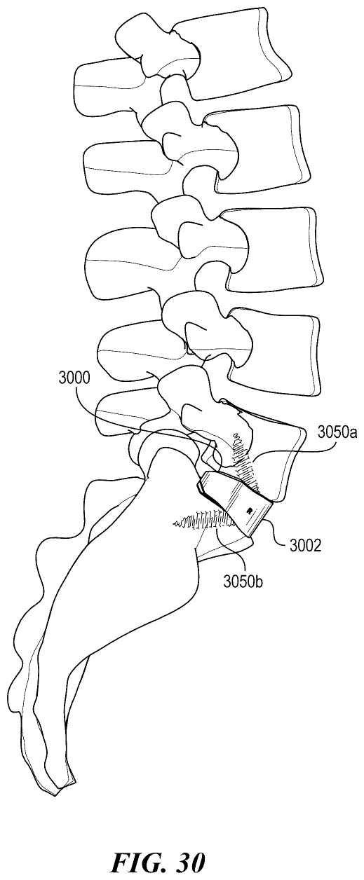

FIG. 8 is a flowchart of a method 800 for performing a surgical procedure in accordance with at least some embodiments of the technology. The method 800 can be used to implant a wide range of implant types. For example, the method 800 can be used or modified to implant fixation systems (e.g., as discussed in connection with FIGS. 30-34), cages (e.g., as discussed in connection with FIGS. 28A-29), and other types of implants. Example surgical steps for implanting a fixation assembly are discussed in connection with FIGS. 9-22. Example steps of method 800 are discussed below with respect to spinal procedures.

At step 802, a patient-specific plate assembly can be positioned in a patient. The patient-specific plate assembly can be positioned using, for example, direct visualization, fluoroscopy, or other imaging techniques. In some embodiments, a robotic surgery system can position the plate assembly in the patient, and a navigation system can confirm positioning prior to performing additional steps.

At step 804, the plate assembly can be temporarily coupled to anatomical elements by one or more spikes or other fixation elements delivered via guides or through holes of the plate. Additionally or alternatively, the fixation plate 130 can include openings for positioning fixation elements. For example, the fixation plate assembly 110 of FIG. 1 can have apertures configured to receive locators (e.g., intra-disc positioners, spikes, or the like). The drill guide elements 132 can be used to perform drilling steps to form drill holes in anatomical elements.

At step 806, the guides can be sequentially or concurrently separated at breakaway connections and removed from the plate. For example, the guides can be removed after being used or after using two or all or more of the guides. For example, if the plate assembly is left in the patient during the drilling procedures, the guides can be removed after use. In other embodiments, the guides can be removed from the patient after drilling the holes. A user can manually remove the guide elements 132 and can then position the fixation plate 130 back in the patient by, for example, aligning the openings 500 with the drill holes.

At step 808, anchors can be passed through the openings 500 and driven into the drill holes, thereby anchoring the fixation plate 130 to the patient's anatomy. The anchors can be bone screws delivered through the plate 130. In some embodiments, the bone screws can have the same configuration. In some embodiments, the screws can have different configurations, including different thread characteristics (e.g., thread pitches, dimensions, etc.), outer diameters, lengths, or the like. The number, configuration, and characteristics of the bone screws can be selected based on the anatomy of the patient, planned fixation, etc.

At step 810, the screws can be optionally locked to the fixation plate 130 by, for example, one or more locking features, such as locking cams or arms. The locking features can be used to hold the head of the screw within recessed regions 610 of the fixation plate 130, prevent rotation of the head, etc.

FIGS. 9-14 show steps of a method for implanting a patient-specific fixation plate assembly configured for implantation along multiple spinal levels in accordance with embodiments of the present technology. FIG. 9 shows anatomy of a patient and the fixation plate assembly 110 positioned along the vertebrae 112, 114, 116. The guide elements 132a, 132b are positioned to help form drill holes in the vertebral body of the vertebra 112. Each of the drill guide elements 132a, 132b defines a trajectory (one indicated in dashed line 910) that is different from the others. The trajectories can be selected to form drill holes in healthy regions of the vertebral body spaced apart from regions prone to fracture and/or failure. The length of the drill guide elements 132a, 132b can be selected to limit distal movement of the drill head 916, thereby limiting the depth of the drill hole.

The drilling process can be repeated for each of the drill guide elements 132 to form drill holes at various locations. The drill holes can be at different positions/orientations relative to one another to provide a customized anchoring solution. A drill bit 918 is positioned in the passageway of the guide element 132b and can be advanced distally, indicated by arrow 924, to form a drill hole 1010 (FIG. 10) in a vertebral body 1012 of the vertebra 112. Optionally, a spike 1018 can be positioned in one of the guides to temporarily fix the fixation plate 130 to the spinal column. In some embodiments, all of the drill guide elements 132 can receive spikes for securely fastening the fixation plate 130 to the spinal column.

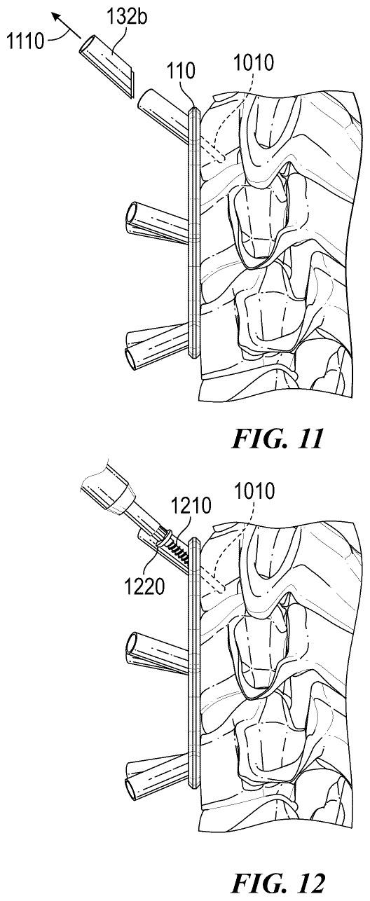

FIG. 11 is a side view of the fixation plate assembly 110 after forming the drill hole 1010. The guide element 132b can be pulled proximally, as indicated by arrow 1110, to break along a breakaway connection. The guide element 132b can be broken off when the fixation plate assembly 110 is inside or outside of the patient. The sequence of drilling holes, removing breakaway features, installing anchors, etc. can be selected based on the procedure.

FIG. 12 shows a screw 1210 being delivered through the fixation plate 130 and into the drill hole 1010. Advantageously, the drill hole 1010 can help guide the screw 1210 along the vertebral body to position the screw 1210 at a desired location. The screw 1210 can include an externally threaded body that is slightly larger than the diameter of the through hole. This allows for a secure fit while limiting or reducing the likelihood of excessive compression or fracturing of the bone. The screw 1210 can have a screw head 1220 configured to cover portions of the breakaway connection that remain connected to the plate. This allows the screw head to protect tissue from contacting the breakaway connection portions. This process can be repeated to implant screws at various locations.

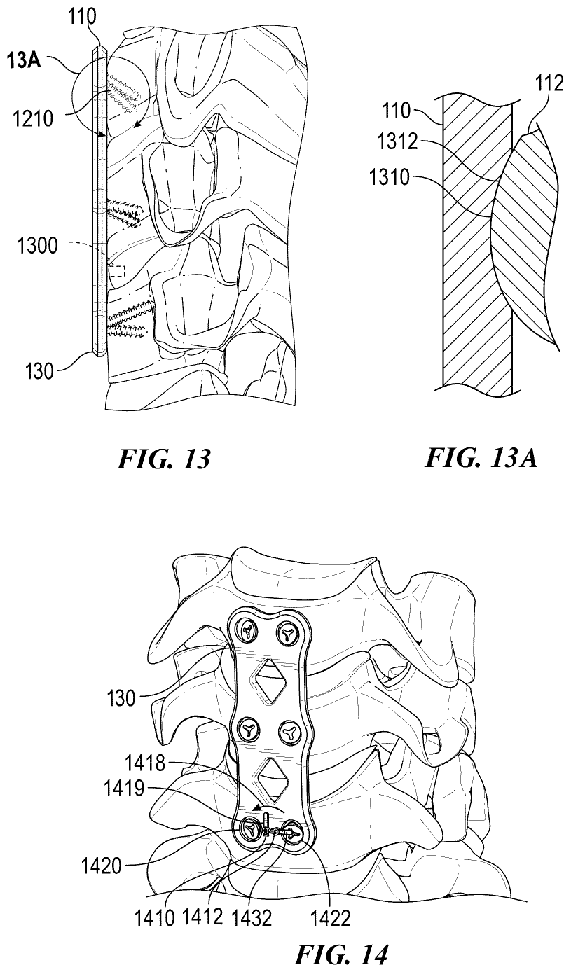

FIG. 13 shows the plate 130 installed with two screws fixing the plate to vertebral bodies. Additional screws can be installed. For example, each vertebra can receive three or more screws holding the plate. This allows for flexibility during surgical procedures. The fixation plate 130 can be contoured to mate with each of the vertebrae. FIG. 13A shows an anatomy-receiving recessed region 1310 configured to receive the sidewall 1312 of the vertebra 112. The guide elements can define a drill bit trajectory for forming a drill hole adjacent to or near anatomy-receiving recessed regions. The fixation plate 130 can include different types of anatomy-receiving topography. Example topography, recesses, projections, and interfaces are described by U.S. Ser. No. 17/100,396, which is disclosed by reference in its entirety.

The fixation plate 130 can be coupled to intervertebral cages by one or more connection mechanisms 1300 (FIG. 13). The connection mechanism 1300 can be rigid or flexible and can be configured to couple the patient-specific positioning feature to the intervertebral cage, to maintain a predetermined configuration of the patient-specific intervertebral implant, when the intervertebral cage is between the vertebrae (e.g., vertebral endplates) and the patient-specific positioning feature contacts the target region. The predetermined configuration can correspond to a target spatial relationship between the cage and positioning feature. The connection mechanisms 1300 can include, without limitation, a rigid connection including one or more screws or bolts (e.g., a lag blot, a carriage bolt, a hex bolt, a machine screw, a wood screw, etc.), a rigid metal arm, etc. Semi-rigid connections can include a semi-rigid metal arm (e.g., a slotted arm, a flexible arm, etc.), an arm with cut-out or bend points, etc. In some embodiments, the connection mechanism 1300 can be a key and slot mechanism, a magnet, a rivet, a tether, or other suitable fastening element(s). Example connection mechanisms, positioning features, patient-specific contouring, and other features are described by U.S. Ser. No. 17/100,396, which is disclosed by reference in its entirety. The implants described by U.S. Ser. No. 17/100,396 can include the features and components disclosed herein.

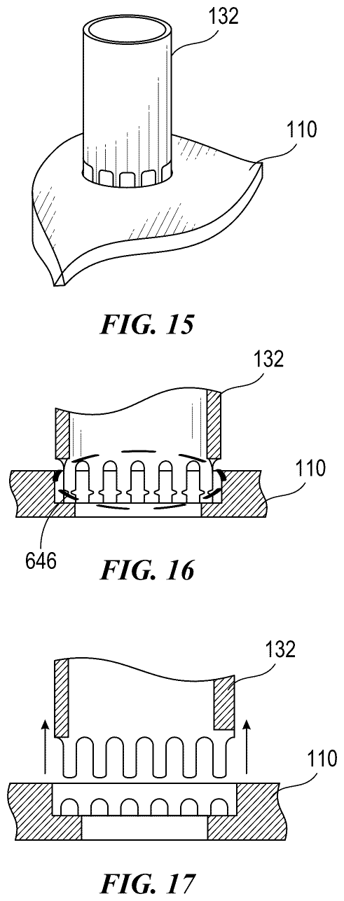

FIG. 14 is an isometric view of the installed fixation plate 130. Locking cams can be used to hold screws. Example locking cams 1410, 1412 can be used to inhibit or prevent backing out of screws 1420, 1422, respectively. For example, the locking cam 1412 can be rotated such that an arm 1432 of the locking cam 1412 extends across the screw head of the screw 1420, thereby holding the screw against the plate. The locking cam 1410 can be rotated, as indicated by arrow 1418, to position an arm 1419 over the head. The configuration of the locking cam can be selected based on the configuration of the breakaway connection. Example locking cams and breakaway connections are discussed in connection with FIGS. 15-22.

FIG. 15 is an isometric view of a breakaway guide element 132 of a fixation plate assembly 110 in accordance with embodiments of the present technology. FIG. 16 is a cross-sectional view of the portion of the patient-specific implant of FIG. 15. FIG. 17 is a cross-sectional view of the portion of the patient-specific implant after the breakaway guide element 132 has been separated from the fixation plate assembly 110. Referring now to FIG. 16, the breakaway guide element 132 can include frangible portions 646 configured to break, fracture, separate, or the like to remove the breakaway guide element 132 from the fixation plate 110. The frangible portions 646 can extend generally parallel to a longitudinal axis of the breakaway guide element 132, an axis of the through hole, etc.

FIG. 18 is a cross-sectional view of a portion of a fixation plate assembly 110 and an angled breakaway guide element 132 in accordance with embodiments of the present technology. The breakaway guide element 132 can include vertical frangible portions 646 that can break to remove the breakaway guide element 132 from the fixation plate 110.

FIG. 19 is a cross-sectional view of a portion of a fixation plate assembly 110 and an angled breakaway guide element 132 in accordance with embodiments of the present technology. The breakaway guide element 132 can include angled frangible portions 646 that can break to remove the breakaway guide element 132 from the fixation plate 110.

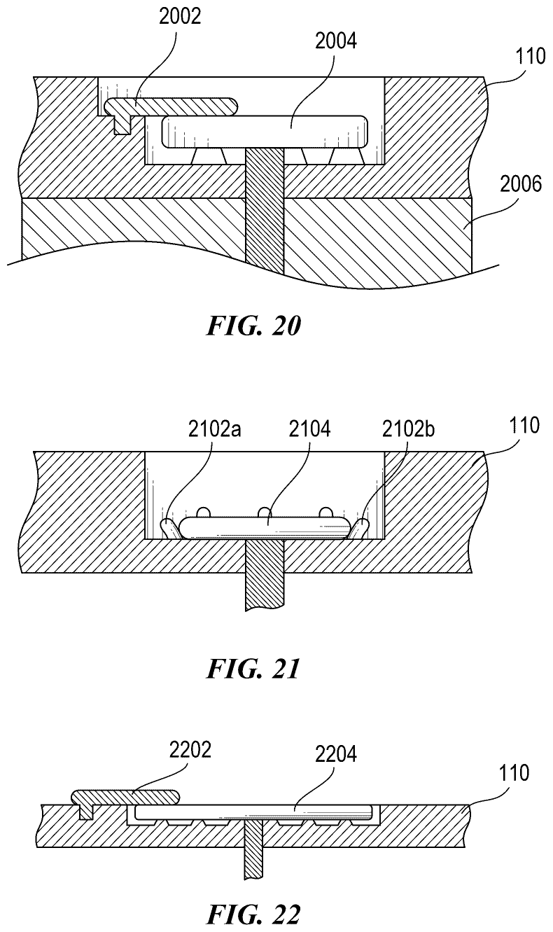

FIGS. 20-22 are cross-sectional views of portions of patient-specific implants with fixation plates and anchors in accordance with embodiments of the present technology. FIG. 21 shows portions 2102a, 2102b of the breakaway connection connected to the fixation plate 110 that are pushed outwardly to snugly hold the outside of the screw head 2104. The portions 2102a, 2102b can be residual portions of the breakaway connection discussed in connection with FIGS. 17 and 18. In some embodiments, the portions 2102a, 2102b can be used with locking cams. FIGS. 20 and 22 also show rotatable locking cams 2002 and 2202 that extend across the screw heads 2004 and 2204 to inhibit, limit, or prevent backing out or rotation of the screw heads 2004, 2204. The position, configuration, and/or operation of the locking cam can be selected based on the component to be locked. One or more anti-rotation elements (e.g., washers, friction elements, etc.), locking elements, washers, and/or other components can be positioned between components, such as between the screw heads and plate of FIGS. 20-22 to inhibit, reduce, or eliminate screw rotation or screw pullout.

### IV. Systems and Methods for Designing, Manufacturing, and Implanting Patient-Specific Implants

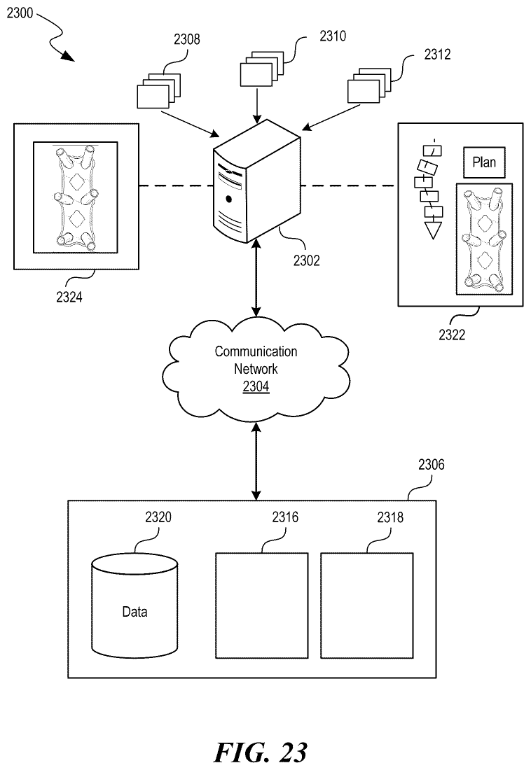

The present technology also provides system and methods for designing and manufacturing patient-specific implants, such as any of the patient-specific implants described herein. FIG. 23 is a network connection diagram illustrating a computing system 2300 for providing patient-specific medical care and configured in accordance with select embodiments of the present technology. The system 2300 is configured to design a patient-specific implant and/or generate a patient-specific surgical plan for a patient. For example, the system 2300 can generate an implant and/or generate a surgical plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, cervical spinal stenosis, or a combination thereof. The surgical plan can include surgical information, surgical plans (e.g., surgical implant procedure, target implant location and/or orientation, etc.), technology recommendations (e.g., device and/or instrument recommendations), and/or medical device designs. For example, the surgical plan can include at least one treatment procedure (e.g., a surgical procedure or intervention) and/or at least one medical device (e.g., an implanted medical device (also referred to herein as an “implant” or “implanted device”) or implant delivery instrument).

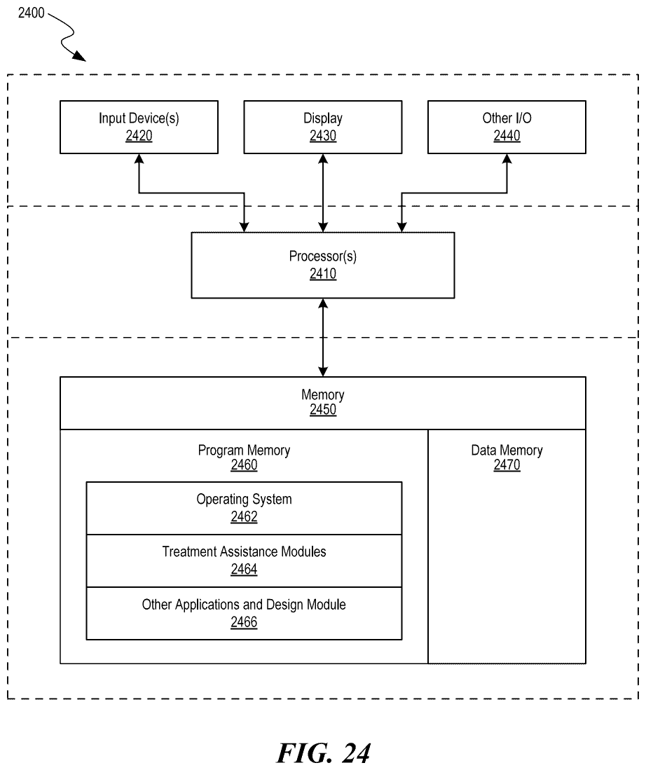

The system 2300 includes a computing device 2302, which can be a user device, such as a smart phone, mobile device, laptop, desktop, personal computer, tablet, phablet, or other such devices known in the art. As discussed in greater detail with reference to FIG. 24, the computing device 2302 can include one or more processors, and memory storing instructions executable by the one or more processors to perform select methods described herein. The computing device 2302 can be associated with a healthcare provider that is treating the patient. Although FIG. 23 illustrates a single computing device 2302, in alternative embodiments, the computing device 2302 can instead be implemented as a computing system encompassing a plurality of computing devices, such that the operations described herein with respect to the computing device 2302 can instead be performed by the computing system and/or the plurality of computing devices.

The computing device 2302 is configured to receive a patient data set 2308 associated with a patient to be treated. The patient data set 2308 can include data representative of the patient's condition, anatomy, pathology, symptoms, medical history, preferences, and/or any other information or parameters relevant to the patient. For example, the patient data set 2308 can include surgical intervention data, treatment outcome data, progress data (e.g., physician notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health-related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-ray images), diagnostic equipment information (e.g., manufacturer, model number, specifications, user-selected settings/configurations, etc.), or the like. In some embodiments, the patient data set 2308 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

In some embodiments, the computing device 2302 can also be configured to receive a surgical team data set 2310. The surgical team data set 2310 can include data representative of the surgical team that will perform the surgery on the patient. For example, the surgical team data set 2310 can include preferences of the surgical team (e.g., preferred implant techniques, preferred implant instruments/tools, etc.), experience of the surgical team (e.g., past procedures performed by the surgical team), scored outcomes of past procedures performed by the surgical team, or the like. As used herein, the term “surgical team” can refer to a group of healthcare practitioners that work together in an operating room during an implant procedure, or to one or more individual surgeons.

In some embodiments, the computing device 2302 can also be configured to receive a facility or provider data set 2312. The facility data set 2312 can include data representative of the facility at which the patient's surgery will occur. For example, the facility data set 2312 can include preferences of the facility (e.g., preferred implant techniques, preferred implant instruments/tools, etc.), experience of the facility (e.g., past procedures performed at the facility), scored outcomes of past procedures performed at the facility, infrastructure available to assist/perform the surgery (e.g., availability of robotic surgical platforms, as well as the type of “input” required to control the “output” of the robotic surgical platforms), or the like. As used herein, the term “facility” can refer to a single operating room facility, a hospital having multiple operating rooms, and/or a network of hospitals.

The computing device 2302 is operably connected via a communication network 2304 to a server 2306, thus allowing for data transfer between the computing device 2302 and the server 2306. The communication network 2304 may be a wired and/or a wireless network. The communication network 2304, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 2306, which may also be referred to as a “treatment assistance network” or “prescriptive analytics network,” can include one or more computing devices and/or systems. As discussed further herein, the server 2306 can include one or more processors, and memory storing instructions executable by the one or more processors to perform the methods described herein. In some embodiments, the server 2306 is implemented as a distributed “cloud” computing system or facility across any suitable combination of hardware and/or virtual computing resources.

The computing device 2302 and/or the server 2306 can design a patient-specific implant (“implant”) based at least in part on the patient data set 2308, the surgical team data set 2310, and/or the facility data set 2312. For example, the server 2306 may include a treatment planning module 2318 that can design, based off of any of the foregoing data inputs, the implant. In some embodiments, the implant includes a design for a vertebral implant including a cage and a plate, such as any of the vertebral implants described with respect to FIGS. 1-7 and 9-22, that are specifically designed to mate with corresponding target anatomical structures. The computing device 2302 and/or the server 2306 can design an interbody device (e.g., a cage), a positioning feature (e.g., a plate), a stabilizing plate, a connection mechanism, and/or one or more fastening elements (e.g., a screw). For example, intervertebral cages can be designed to produce an anatomical correction (e.g., a targeted spinal alignment). Before, while, or after designing the intervertebral cages, a plate can be designed to match anatomical features in the anatomical correction. In some embodiments, to design the connection mechanism, the target position of the cage can be determined by analyzing images of the patient. The patient spine can be analyzed to identify a suitable target site for the positioning feature. For example, the vertebral bodies can be analyzed to identify regions with suitable geometry and mechanical properties to interface with a locking plate. The computing device 2302 and/or the server 2306 can design the connection mechanism based on a level of movement (e.g., a maximum level of relative movement) between the cage and the locking plate. In spinal fusion procedures, the connection mechanism can be a rigid arm that substantially prevents relative movement between the cage and the plate. Example connection mechanisms, cages, and plates are disclosed in U.S. application Ser. No. 17/100,396, which is incorporated by reference. The target spatial relationship between the components of the implant can be determined based on the patient data and targeted outcome.

Additional implants include, but are not limited to, screws (e.g., bone screws, spinal screws, pedicle screws, facet screws), other interbody implant devices, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.).

The implant can be designed to match the patient's existing anatomy. For example, the implant can be designed such that various surfaces of the implant mate with corresponding surfaces of patient anatomy, as previously described. In some embodiments, the implant can be designed to provide a correction to the patient's existing anatomy in addition to mating with one or more surfaces of the patient anatomy. For example, the treatment planning module 2318 may analyze image data of the patient's native anatomy to determine whether an anatomical correction is needed. The image data may show the patient's native anatomical configuration (e.g., pre-operative anatomy), such as the geometry, orientation, and topography of various anatomical features. In some embodiments, for example, the image data may show (and/or be used to determine) various anatomical characteristics, including, but not limited to, vertebral spacing, vertebral orientation, vertebral translation, abnormal bony growth, abnormal joint growth, joint inflammation, joint degeneration, tissue degeneration, stenosis, scar tissue, lumbar lordosis, Cobb angle(s), pelvic incidence, disc height, segment flexibility, rotational displacement, and other spinal tissue characteristics. If an anatomical correction is not required, the treatment planning module 2318 can design the implant to fit the patient's native anatomy. If an anatomical correction is required, the treatment planning module 2318 can design the implant such that, when the implant is implanted in the patient, it provides the anatomical correction. Additional details for designing patient-specific implants to provide one or more desired anatomical corrections can be found in U.S. application Ser. No. 16/987,113, filed Aug. 6, 2020, the disclosure of which is incorporated by reference herein in its entirety.

In some embodiments, the generated implant design is a design for an entire device. Alternatively, the generated design can be for one or more components of a device (e.g., a plate or a cage), rather than the entire device. In some embodiments, the implant design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The implants described herein are expected to improve delivery into the patient's body, placement at the treatment site, and/or interaction with the patient's anatomy.

The computing device 2302 and/or the server 2306 can also design a patient-specific surgical plan (“surgical plan”) based on the patient data set 2308, the surgical team data set 2310, and/or the facility data set 2312. The surgical plan can include a detailed procedure for implanting the implant to a specific target position within the patient. For example, the surgical plan can include aspects of a pre-operative plan (e.g., detection and measurement of patient's anatomy, preparation of patient for a surgical procedure, etc.), a surgical procedure, a surgical approach (e.g., implant technique), one or more surgical steps (preparing tissue for an incision, making an incision, making a resection, removing tissue, manipulating tissue, performing a corrective maneuver, delivering the implant to a target site, deploying the implant at the target site, adjusting the implant at the target site, manipulating the implant once it is implanted, securing the implant at the target site, explanting the implant, suturing tissue, etc.) a target position, site, or location of the implant (e.g., a location, orientation, etc.), and other aspects related to pre-operative, operative, or post-operative plans.

In some embodiments, the surgical plan includes an orthopedic surgical procedure such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). Spinal surgery can also include non-fusion surgeries, such as artificial disc replacements. In some embodiments, the surgical procedure includes descriptions of and/or instructions for performing one or more aspects of a patient-specific surgical procedure. For example, the surgical procedure can include one or more of a surgical approach, a corrective maneuver, or a bony resection.

In some embodiments, the surgical plan includes a target position of the implant. In some embodiments, the surgical plan optionally includes a recommendation to remove tissue to clear space for the implant at the target position. For example, the surgical plan may include instructions to perform an osteotomy, muscular resection, soft tissue detachment, soft tissue retraction, or the like to prepare the patient to receive the patient-specific implant. In some embodiments, the surgical plan includes a manipulation of tissue to prepare the patient to receive the implant. For example, the surgical plan may include instructions to adjust a relative position of two vertebrae, increase a distance between two vertebrae, or the like.

In some embodiments, the surgical plan includes machine-readable instructions for carrying out various steps of the surgical plan. The machine-readable instructions can be configured such that, when executed by a surgical robotic platform, the machine-readable instructions cause the surgical robotic platform to execute various aspects of an operative procedure associated with implanting the implant. For example, the surgical platform may prepare tissue for an incision, make an incision, make a resection, remove tissue, manipulate tissue, perform a corrective maneuver, deliver the implant to a target site, deploy the implant at the target site; adjust a configuration of the implant at the target site, manipulate the implant once it is implanted, secure the implant at the target site, explant the implant, suture tissue, and the like. The instructions may therefore include particular instructions for articulating robotic arms, instruments, and/or tools to perform or otherwise aid in the delivery of the patient-specific implant.

In some embodiments, the surgical plan includes step-by-step written, verbal, and/or graphic instructions that show a surgeon how to perform the patient-specific surgical plan. The patient-specific surgical plan can be displayed to the surgeon before and/or during the operative procedure (e.g., via display 2322). In some embodiments, the written, verbal, and/or graphic instructions can be encoded in computer-readable instructions. The encoded instructions can be decoded and displayed to the surgeon before and/or during the operative procedure. In some embodiments, the patient-specific surgical plan includes both machine-readable instructions and written, verbal, and/or graphic illustrations.

In some embodiments, the system 2300 may consider one or more reference data sets when designing the patient-specific implant and/or the patient-specific surgical plan. For example, in some embodiments the server 2306 includes at least one database 2320 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 2320 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, preferences, and/or any other information or parameters relevant to the reference patient, such as any of the data described herein with respect to the patient data set 2308. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one treatment procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, activity level, return to work, complications, recovery times, efficacy, mortality, and/or follow-up surgeries.

In some embodiments, the server 2306 receives at least some of the reference patient data sets from a plurality of healthcare provider computing systems. Each healthcare provider computing system can include at least one reference patient data set (e.g., reference patient data sets) associated with reference patients treated by the corresponding healthcare provider. The reference patient data sets can include, for example, kinematic records, electronic medical records, electronic health records, biomedical data sets, etc.

In embodiments in which the implant and/or the surgical plan is designed based on the reference data, the data analysis module 2316 can include one or more algorithms for identifying a subset of reference data from the database 2320 that is likely to be useful in developing a treatment plan. For example, the data analysis module 2316 can compare patient-specific data (e.g., the patient data set 2308 received from the computing device 2302) to the reference data from the database 2320 (e.g., the reference patient data sets) to identify similar data (e.g., one or more similar patient data sets in the reference patient data sets). The comparison can be based on one or more parameters, such as age, gender, BMI, pathology, kinematics, lumbar lordosis, pelvic incidence, and/or treatment levels. The parameter(s) can be used to calculate a similarity score for each reference patient. The similarity score can represent a statistical correlation between the patient data set 2308 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients. In some embodiments, the data analysis module 2316 includes one or more algorithms that select a set or subset of the reference patient data based on criteria other than patient parameters, such as the surgical team data set 2310 (e.g., based on surgeon expertise, outcomes of particular types of procedures performed by the surgeon, etc.) and/or the facility data set 2312 (e.g., surgical equipment such as surgical robots).

The data analysis module 2316 can further be configured with one or more algorithms to select a subset of the reference patient data sets, e.g., based on similarity to the patient data set 2308 and/or treatment outcome of the corresponding reference patient. For example, the data analysis module 2316 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, range of motion, kinematic data, HRQL, activity level, complications, recovery times, efficacy, mortality, or follow-up surgeries. As described in further detail below, in some embodiments, the data analysis module 2316 calculates an outcome score by assigning values to each outcome parameter. A patient can be considered to have a favorable outcome if the outcome score is above, below, or at a specified threshold value.

In some embodiments, the data analysis module 2316 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 2316 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular healthcare provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific treatment plan to perform a battlefield surgery can be based on reference patient data from similar battlefield surgeries and/or datasets associated with battlefield surgeries. In another example, the patient-specific treatment plan can be generated based on available robotic surgical systems. The reference patient data sets can be selected based on patients that have been operated on using comparable robotic surgical systems under similar conditions (e.g., size and capabilities of surgical teams, hospital resources, etc.).

In embodiments in which the implant and/or the surgical plan is designed based on the reference data, the treatment planning module 2318 can include one or more algorithms that generate the implant and/or the surgical plan based on the reference data. In some embodiments, the treatment planning module 2318 is configured to develop and/or implement at least one predictive model for generating the treatment plan, also known as a “prescriptive model.” The predictive model(s) can be developed using clinical knowledge, statistics, machine learning, artificial intelligence (Al), neural networks, or the like. In some embodiments, the output from the data analysis module 2316 is analyzed (e.g., using statistics, machine learning, neural networks, Al, etc.) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a treatment plan will produce a favorable outcome for the particular patient. The predictive model(s) can be validated, e.g., by inputting data into the model(s) and comparing the output of the model to the expected output.

In some embodiments, the treatment planning module 2318 is configured to generate the implant design based on previous treatment data from reference patients. For example, the treatment planning module 2318 can receive a selected subset of reference patient data sets and/or similar patient data sets from the data analysis module 2316, and determine or identify treatment data from the selected subset. The treatment data can include, for example, range of motion and/or other kinematic data, treatment procedure data (e.g., surgical procedure or intervention data) and/or medical device design data (e.g., implant design data) that are associated with favorable or desired treatment outcomes for the corresponding patient. The treatment planning module 2318 can analyze the treatment procedure data and/or medical device design data to determine an optimal treatment protocol for the patient to be treated. For example, the treatment procedures and/or medical device designs can be assigned values and aggregated to produce a treatment score. The patient-specific treatment plan can be determined by selecting treatment plan(s) based on the score (e.g., higher or highest score; lower or lowest score; score that is above, below, or at a specified threshold value). The personalized treatment plan can be based on, at least in part, the patient-specific technologies or patient-specific selected technology.

Alternatively or in combination, the treatment planning module 2318 can generate the implant designs based on correlations between data sets. For example, the treatment planning module 2318 can correlate implant designs and medical device design data from implant designs for similar patients with favorable outcomes (e.g., as identified by the data analysis module 2316). Correlation analysis can include transforming correlation coefficient values to values or scores. The values/scores can be aggregated, filtered, or otherwise analyzed to determine one or more statistical significances. These correlations can be used to determine treatment procedure(s) and/or medical device design(s) that are optimal or likely to produce a favorable outcome for the patient to be treated.

Alternatively or in combination, the treatment planning module 2318 can generate designs using one or more Al techniques. Al techniques can be used to develop computing systems capable of simulating aspects of human intelligence, e.g., learning, reasoning, planning, problem solving, decision making, etc. Al techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 2318 generates the treatment plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 2320, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate the treatment plan, the patient data set 2308, the surgical team data set 2310, and/or the facility data set 2312 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one treatment plan for the patient. In embodiments where multiple trained machine learning models are used, the models can be run sequentially or concurrently to compare outcomes and can be periodically updated using training data sets. The treatment planning module 2318 can use one or more of the machine learning models based the model's predicted accuracy score.

The implant design and/or the surgical plan generated by the treatment planning module 2318 can be transmitted via the communication network 2304 to the computing device 2302 for output to a user (e.g., clinician, surgeon, healthcare provider, patient). In some embodiments, the computing device 2302 includes or is operably coupled to a display 2322. The display 2322 can show various aspects of a surgical procedure to be performed on the patient, such as the surgical approach, treatment levels, corrective maneuvers, tissue resection, and/or implant placement. To facilitate visualization, a virtual model of the surgical procedure can be displayed. Additionally or alternatively, the display 2322 can show a design for the implant, such as a two-or three-dimensional model of the device design. The display 2322 can also show patient information, such as two-or three-dimensional images or models of the patient's anatomy where the surgical procedure is to be performed and/or where the device is to be implanted. The display 2322 can also display structural features of the implant suitable for contacting anatomical features to improve treatment, reduce implant movement, etc. The structural features can be rigid surfaces (e.g., outer surfaces of an implant body), anchors, fixation features, etc. Images of the implantation site can be analyzed to identify such anatomical features identified by the treatment planning module 2318. The computing device 2302 can further include one or more user input devices (not shown) allowing the user to modify, select, approve, and/or reject the displayed treatment plan(s).

In some embodiments, the patient-specific implant design generated by the treatment planning module 2318 can be transmitted from the computing device 2302 and/or the server 2306 to a manufacturing system 2324 for manufacturing a corresponding medical device. The manufacturing system 2324 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

The manufacturing system 2324 can manufacture all or some of the components of a kit, a surgical tray, etc. The components can be selected based on requirement(s), including regulatory requirements, reimbursement requirements, or other requirements. Kits can include one or more implants, instruments, instructions for use, and reusable and disposable components. The kit requirements can be retrieved from a database. The system 2300 can synchronize the surgical plan with the requirements to generate patient-specific surgical kits meeting the requirements.

Various types of manufacturing systems are suitable for use in accordance with the embodiments herein. For example, the manufacturing system 2324 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 2324 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 2324 can manufacture one or more patient-specific medical devices based on fabrication instructions or data (e.g., CAD data, 3D data, digital blueprints, stereolithography data, or other data suitable for the various manufacturing technologies described herein). In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The treatment plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 2318 generates control instructions configured to cause a surgical robot (e.g., robotic surgery systems, navigation systems, etc.) to partially or fully perform a surgical procedure. The control instructions can be transmitted to the robotic apparatus by the computing device 2302 and/or the server 2306.

Following the treatment of the patient in accordance with the treatment plan, treatment progress can be monitored over one or more time periods to update the data analysis module 2316 and/or treatment planning module 2318. Post-treatment data can be added to the reference data stored in the database 2320. The post-treatment data can be used to train machine learning models for developing patient-specific treatment plans, patient-specific medical devices, or combinations thereof.

It shall be appreciated that the components of the system 2300 can be configured in many different ways. For example, in alternative embodiments, the database 2320, the data analysis module 2316, and/or the treatment planning module 2318 can be components of the computing device 2302, rather than the server 2306. As another example, the database 2320, the data analysis module 2316, and/or the treatment planning module 2318 can be located across a plurality of different servers, computing systems, or other types of cloud-computing resources, rather than at a single server 2306 or computing device 2302.

Additionally, in some embodiments, the system 2300 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like. In some embodiments, the system 2300 may include additional features and/or capabilities, such as any of those described in U.S. application Ser. No. 16/735,222, filed Jan. 6, 2020, the disclosure of which is incorporated by reference herein in its entirety.

FIG. 24 illustrates a computing device 2400 suitable for use in connection with the system 2300 of FIG. 23, according to an embodiment. The computing device 2400 can be incorporated in various components of the system 2300 of FIG. 23, such as the computing device 2302 or the server 2306. The computing device 2400 includes one or more processors 2410 (e.g., CPU(s), GPU(s), HPU(s), etc.). The processor(s) 2410 can be a single processing unit or multiple processing units in a device or distributed across multiple devices. The processor(s) 2410 can be coupled to other hardware devices, for example, with the use of a bus, such as a PCI bus or SCSI bus. The processor(s) 2410 can be configured to execute one more computer-readable program instructions, such as program instructions to carry out any of the methods described herein.

The computing device 2400 can include one or more input devices 2420 that provide input to the processor(s) 2410, e.g., to notify it of actions from a user of the computing device 2400. The actions can be mediated by a hardware controller that interprets the signals received from the input device and communicates the information to the processor(s) 2410 using a communication protocol. Input device(s) 2420 can include, for example, a mouse, a keyboard, a touchscreen, an infrared sensor, a touchpad, a wearable input device, a camera-or image-based input device, a microphone, or other user input devices.

The computing device 2400 can include a display 2430 used to display various types of output, such as text, models, virtual procedures, surgical plans, implants, graphics, and/or images (e.g., images with voxels indicating radiodensity units or Hounsfield units representing the density of the tissue at a location). In some embodiments, the display 2430 provides graphical and textual visual feedback to a user. The processor(s) 2410 can communicate with the display 2430 via a hardware controller for devices. In some embodiments, the display 2430 includes the input device(s) 2420 as part of the display 2430, such as when the input device(s) 2420 include a touchscreen or is equipped with an eye direction monitoring system. In alternative embodiments, the display 2430 is separate from the input device(s) 2420. Examples of display devices include an LCD display screen, an LED display screen, a projected, holographic, or augmented reality display (e.g., a heads-up display device or a head-mounted device), and so on.

Optionally, other input/output (I/O) devices 2440 can also be coupled to the processor(s) 2410, such as a network card, video card, audio card, USB, firewire or other external device, camera, printer, speakers, CD-ROM drive, DVD drive, disk drive, or Blu-ray device. Other I/O devices 2440 can also include input ports for information from directly connected medical equipment such as imaging apparatuses, including MRI machines, X-ray machines, CT machines, etc. Other I/O devices 2440 can further include input ports for receiving data from these types of machines from other sources, such as across a network or from previously captured data, for example, stored in a database.

In some embodiments, the computing device 2400 also includes a communication device (not shown) capable of communicating wirelessly or wire-based with a network node. The communication device can communicate with another device or a server through a network using, for example, TCP/IP protocols. The computing device 2400 can utilize the communication device to distribute operations across multiple network devices, including imaging equipment, manufacturing equipment, etc.

The computing device 2400 can include memory 2450, which can be in a single device or distributed across multiple devices. Memory 2450 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 2450 is a non-transitory computer-readable storage medium that stores, for example, programs, software, data, or the like. In some embodiments, memory 2450 can include program memory 2460 that stores programs and software, such as an operating system 2462, one or more treatment assistance modules 2464, and other application programs and/or design modules 2466. The treatment assistance module(s) 2464 can include one or more modules configured to perform the various methods described herein (e.g., the data analysis module 2316 and/or treatment planning module 2318 described with respect to FIG. 23). Memory 2450 can also include data memory 2470 that can include, e.g., reference data, configuration data, settings, user options or preferences, etc., which can be provided to the program memory 2460 or any other element of the computing device 2400.

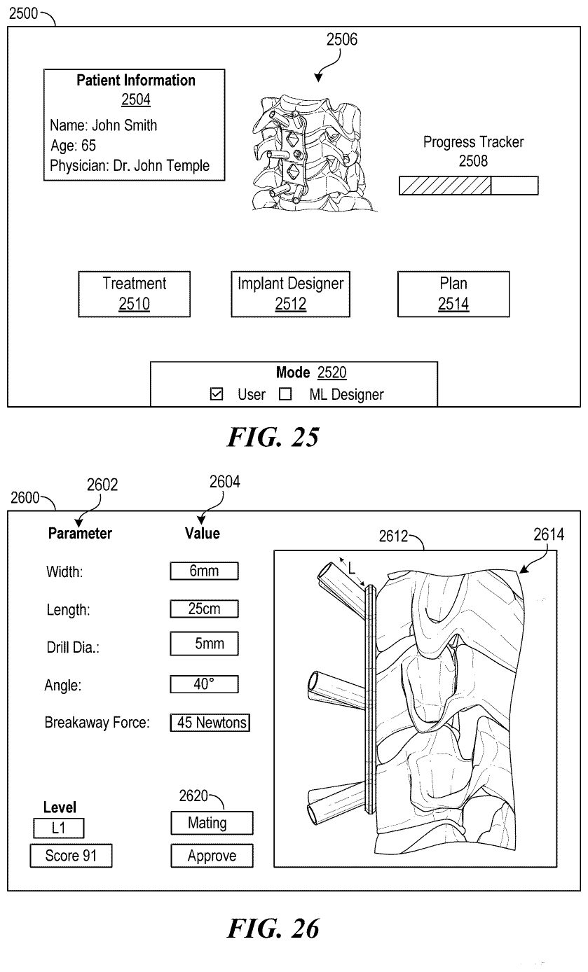

FIG. 25 illustrates a surgery manager system user interface 2500 for selecting treatments, designing implants, and/or managing plans, according to at least some embodiments. The user interface 2500 can show a planned corrected anatomy of the patient. The user interface 2500 can include a patient information window 2504 with patient information. The patient information can include, without limitation, the patient's biometrics, age, health status, electronic medical records, physician information, or other information disclosed herein. A user can select information to be displayed in the patient information window 2504 to assist with treatment planning.

The user interface 2500 can include viewable anatomy information 2506. The anatomy information 2506 can include, without limitation, an image of patient anatomy, patient images, virtual models (e.g., virtual models of anatomy, virtual models of implants, etc.), or the like. A user can select the anatomy information to be displayed. The viewable anatomy information 2506 can include pre-operative models, intra-operative models, post-operative models, implant models, instrument models, or the like.

The user interface 2500 can include a progress tracker 2508 indicating progress of treatment planning, implant design, treatment plan generation, or the like. In some embodiments, the progress tracker 2508 indicates a percentage completion of treatment planning and approval. This can help a physician when scheduling a surgical procedure. In some embodiments, the progress tracker 2508 indicates a percentage completion of implant designing.

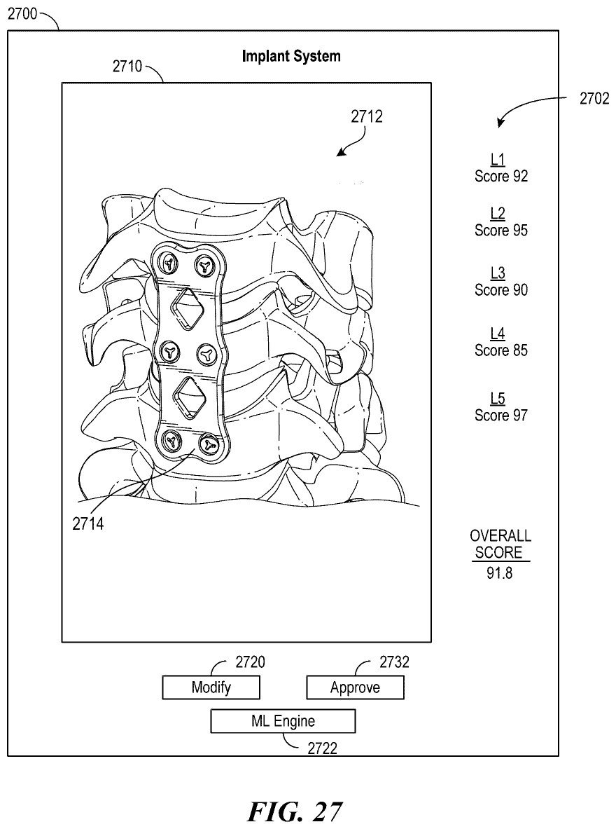

The user interface 2500 can include a treatment selector 2510 for selecting a candidate treatment as discussed herein, an implant designer selector 2512 for launching an implant designer platform as discussed in connection with FIGS. 26 and 27, and a plan selector 2514 for selecting a treatment plan as discussed herein. The user interface 2500 can include a mode selector 2520 for selecting a design mode. In some embodiments, a design platform includes a user design mode and a machine learning (ML) designer mode. A user can select a user design mode (illustrated as selected in FIG. 25) to allow a user to input values for designing implants, generating surgical plans, or the like. The ML designer mode can be selected to have the system design platform use one or more machine learning modules to generate at least a portion of the treatment. For example, a user can generate an initial design using the ML designer. A user can select a user mode for modifying the initial design. The ML designer can be selected again to generate predicted outcomes of the modified design. The ML designer can design implants or features thereof, as discussed in connection with FIGS. 26-31.

The user interface 2500 can be used to individually, collectively, concurrently, sequentially, and/or iteratively design implants. Example design process flows include individual design flows, collective design flows, concurrent design flows, sequential design flows, and/or iterative design flows. In individual design flows, a user can select implants (or features) to be designed one at a time. The design process can be updated to reflect completed designs. In collective design flows, a user can select a group of implants (or features) to be collectively designed at the same time or sequentially. In collective design flows, a user can select a group of implants (or features) to be collectively designed at the same time. In concurrent design flows, a user can select design implants (or features) to be designed at the same time (e.g., using the same virtual model of anatomy). In sequential design flows, a user can select an order to design implants. The design process can be adjusted to reflect completed designs. In iterative design flows, a user can select design implants (or features) to be iteratively designed using, for example, ML models, computer aided design software, finite element analysis software, etc. For example, a set of intervertebral cages can be concurrently designed to achieve an anatomical correction. After designing the intervertebral cages, a set of fixation plates can be designed to hold anatomical features in the corrected configuration. In some procedures, a posterior fixation system and a set of intervertebral cages can be concurrently designed to achieve an anatomical correction. Ancillary components (e.g., a set of fixation plates, a set of screws, a set of anchors, instruments, etc.) can be designed for installing the posterior fixation system, cages, etc. A user can select the design process flow for a design process and compare output from different design process flows.

FIG. 26 illustrates a designer graphical user interface 2600, in accordance with at least some embodiments. The user interface 2600 includes parameters 2602, values 2604, and a plate designer window 2612. The parameters 2602 can include, without limitation, dimensions, surface information, angle information, thread information (e.g., pitch), breakaway features, or the like. The values can include, for example, width or diameter of passageways, diameters of breakaway features (e.g., inner diameters, outer diameters, etc.), lengths, angles, depth of recessed regions, or the like. Angle information can include, for example, the angle between the longitudinal axis of a bone screw and another feature, such as a face plate. The user interface 2600 can include a mating feature selector 2620 for selecting the configuration of a mating feature. For example, the mating feature can be non-barbed, barbed, or have another configuration. The level information can indicate the level of the anchor. For example, the user interface 2600 indicates that the anchor is designed for the L1 level. The designer window 2612 can include a model 2614 of the plate, an anatomical model, etc. The parameters can be identified to assist with the planning and/or design process. A user can select the parameter(s) (e.g., length “L” in FIG. 26) in the anchor designer window 2612 to be updated and insert the new values in the value boxes.

The designer window 2612 can display planned corrections, intraoperative surgical steps, disease progression, etc. In the illustrated embodiment, the anatomical images can be based on an image (e.g., X-ray, CT scan) of the patient with an overlaid image of the plate, screws, etc. This allows the physician or user to evaluate how the implant will be positioned with respect to anatomical elements of the patient. The interfaces and windows can display any number and type of images disclosed herein. For example, the user can select a window and then select the types and number of images to be displayed. A user can select the displayed parameters and components to redesign or modify those parameters or components.

FIG. 27 illustrates an implant system designer graphical user interface 2700, in accordance with at least some embodiments. The user interface 2700 can include scores 2702, parameters, values, and an implant system designer window 2710 that shows a model 2712 of the patient's anatomy and an implant system 2714. Each region can be individually scored to assess different portions of the implant system 2714. For example, the L2 score of 95 is greater than the L1 score 92. A user can modify the design's implants or components to increase the L1 score, thereby increasing the overall score. For example, if the L1 score of 92 is increased, the overall score can be increased to be greater than the illustrated 91.8. A user can select to modify input 2720 to modify a portion of the implant system 2714. The user can select a region to be modified using a box, pointer, touchscreen, or other input. For example, as a user modifies a drill guide model of an implant, the user interface can generate/display a viewable image of the model of the implant system with the modified drill guide. A user can then modify the implant using an implant design platform, as discussed in connection with FIGS. 1-24. The score for the respective components can be dynamically updated to provide real-time feedback. A user can evaluate the position of the anatomy achieved by the implant by viewing the model.

A user can select a machine learning (ML) engine input 2722 to perform at least a part of the design process using a machine learning module. For example, a user can select one or more levels to be designed using the machine learning module. The user can select other levels for manual design of components, which can be checked or modified using the machine learning engine. The user can select an approve input 2732 to approve the implant design. In some embodiments, the system can require that overall scores be equal to or greater than an acceptable score before allowing for user approval when the implant design is predicted to achieve at least a threshold outcome.

The user interface 2700 may allow for seamless, automated switching between different modes, such as a user design mode and an ML design mode for creating patient-specific implants. In the user design mode, a healthcare provider may manually input or adjust parameters for the implant design based on their expertise and preferences. The ML design mode, on the other hand, may utilize trained machine learning algorithms to automatically generate or optimize implant designs based on patient data, treatment goals, and historical outcomes. Users may have the flexibility to initiate the design process in either mode and switch between modes at any point during the design workflow. For example, a user may start with an ML-generated design and then switch to user mode to make fine-tuned adjustments, or vice versa. This hybrid approach may allow for leveraging both the efficiency of ML algorithms and the nuanced judgment of experienced clinicians in creating optimized patient-specific implants. The system may also provide real-time feedback and updated scores as designs are modified in either mode, helping guide users toward implant designs that may best meet the patient's needs and treatment objectives. In some embodiments, the system can switch modes based on one or more design triggers. The design triggers can include, without limitation, parameters of models being outside of an acceptable range, within a trigger range, etc. The ranges can be set by a user, the ML system, a healthcare provider, etc. For example, if a design is predicted to not meet acceptable design criteria (e.g., predicted to generate an unacceptable outcome, not meet regulatory requirements, etc.), the system can automatically switch the system to an ML compliance design mode to modify the design to meet acceptable design criteria.

In some embodiments, the system can have a third-party design mode in which the system can determine one or more parameters of a model for which additional information is needed. The system can query third-party databases (e.g., remote servers) to obtain information associated with the implant. The system can acquire the information and then integrate it into the model. For example, the system can design customized features of an implant system and can obtain recommended dimensions of third-party customized components, dimensions of standard components (e.g., pedicle screws, bone anchors, intervertebral bodies, etc.), or the like. This allows the system to generate customized implant systems that incorporate other manufacturers' customized components, standard components, or the like. In some embodiments, the system can obtain information from literature databases to determine parameters for implants. The number and type of design modes, design flows, and switching mode triggers can be selected based on the user-inputted design goals. Example implants that can be designed are discussed in connection with FIGS. 28A-34.

FIG. 28A is an isometric view of a representative patient-specific spinal fusion device 2800 configured in accordance with select embodiments of the present technology. FIG. 28B is a side view of the device 2800 of FIG. 28A showing the breakaway guide element 132g coupled to the device 2800. The device 2800 can be a patient-specific interbody implant. The device 2800 can be implanted in a patient's spine to promote fusion of two or more vertebral bodies. In particular, the device 2800 can be positioned within a disc space between two vertebral endplates (e.g., after a discectomy). The device 2800 includes breakaway guide elements 132g and 132h (e.g., tubular members, elongate fingers, etc.) coupled to the device 2800 (similar to breakaway guide elements 132 as described in connection with FIGS. 1-22). FIG. 29 is an isometric view of the device 2800 of FIG. 28A after the breakaway guide elements 132g and 132h have been removed from the device 2800.

FIG. 30 is a side (e.g., lateral) view of a device 3000 implanted in the patient's spine. The device 3000 can also include a first fixation element 3050a and a second fixation element 3050b. The fixation elements 3050a and 3050b can anchor an implant 3002 to the patient's spine. The device 3000 is shown as implanted in the sacral/lumbar region of the patient's spine, e.g., between L5 and S1. In other embodiments, the device 3000 can be implanted at other vertebral levels, including other lumbar levels, thoracic levels, or cervical levels. As one skilled in the art will appreciate, FIG. 30 is provided as an example only, and the device described herein can be implanted at any vertebral level in the patient's spine to promote intervertebral fusion. An optional fixation plate can be implanted to further stabilize the spine and can be connected to the device 3000.

FIG. 31 illustrates a fixation system 3110 that has been placed along the lumbar spine. The fixation system 3110 can be used to treat patients who have, for example, a degenerative or deformative condition of the spine. Rods 3150 can be designed to accommodate anatomy and can have breakaway guides. In one embodiment, the rods 3150 (one identified) can be fixed to the spine using bone screws 3100, 3102, 3104. Unique shapes or configurations of the rods 3150 can accommodate individual patient anatomy or mechanical performance. The rod 3150 can additionally, or alternatively, be configured to provide fixation to the sacrum and/or ilium. Sacral screw 3102 and iliac screw 3100 can provide fixation to areas outside of the spine. Surgeons using traditional fixation devices (rods, screws, connectors, etc.) recognize that bending a rod to the appropriate shape for iliac fixation is difficult and often results in the use of supplemental devices that are difficult to use.

Locking elements 3113 and 3152 are configured to secure bone screws 3100, 3102, 3104 to the rod 3150. Longitudinal element 3154 can be configured to (1) avoid anatomy (e.g., have a particular shape), (2) engage anatomy (e.g., grasp a particular landmark or feature, including a process or lamina), (3) have a desired mechanical performance (e.g., have particular dimensions or shape), and/or (4) provide a biological response (e.g., have a particular surface configuration or a particular coating). In one embodiment, locking element 3113 can be configured to provide limited relative movement between bone screw 3100, 3102, 3104 to encourage fusion or avoid causing or worsening adjacent segment disease (proximal junctional kyphosis). In one embodiment, locking element 3113 acts to expand a spherical feature on the proximal end of bone screw 3100, 3102, 3104. In one embodiment, locking element 3113 and longitudinal element 3154 can be configured to have mating features that constrict or control relative motion between components. Mating features can include teeth, grooves, bosses, reliefs, and surface finishes to allow for interdigitation and mechanical engagement between components.

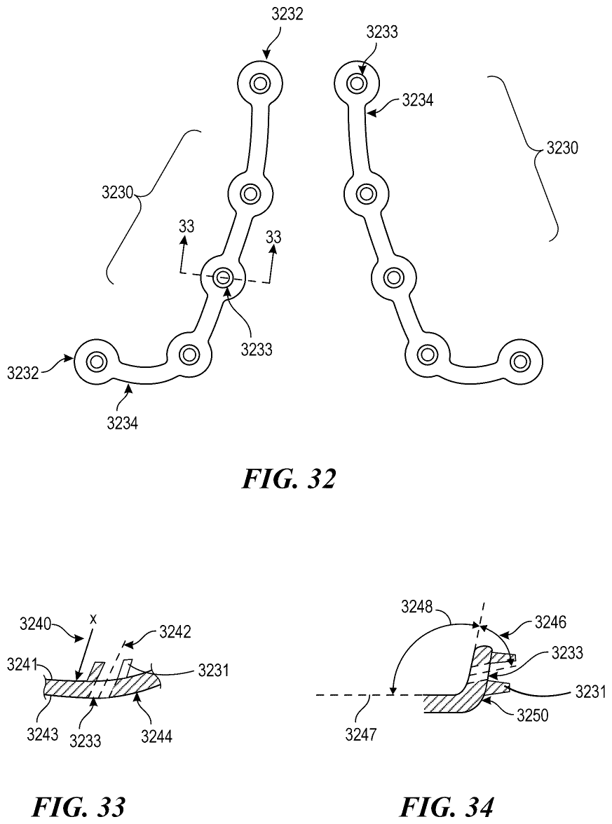

FIG. 32 illustrates the fixation system in accordance with an embodiment of the present invention. Fixation rod or plate 3230 is comprised of longitudinal segments 3234 and nodes 3232. Nodes 3232 have features that provide for receiving of a fixation element or bone screw. The length, orientation, and shape of plate 3230, longitudinal segments 3234, and nodes 3232 can be determined by using surgical planning software.

FIGS. 33 and 34 show breakaway guides 3231 and a hole 3233 that allows for receiving of a bone screw. Breakaway guide 3231 and/or holes 3233 may be oriented orthogonal to a top or bottom surface. Alternatively (FIG. 33), hole axis 3242 may be oriented at an angle relative to a top or bottom surface (e.g., oriented at a non-orthogonal angle). Top surface 3241 and/or bottom surface 3243 may be curved or described by a radius 3240. Top surface 3241 is shown in FIG. 33 to be concave and bottom surface 3243 is shown as convex, but in other embodiments, bottom surface 3243 may be concave and top surface 3241 may be convex. In still other embodiments, one or both of top surface 3241 and bottom surface 3243 may include one or more convex portions and one or more concave portions. Top and bottom surfaces 3241, 3243 may be configured with features to provide improved locking between adjacent components (e.g., mating features including teeth, bumps, recesses). Top and bottom surfaces 3241, 3243 may have a unique shape with features 3244 that provide for accommodation of anatomical features. Top and bottom surfaces 3241, 3243 may be angled relative to one another. Angle 3248, angle 3246, or arc 3250 of FIG. 34 can be used to describe relationships between sections of top and bottom surfaces 3241, 3243. In some instances, node 3232 can be positioned directly lateral to adjacent node 3232. Longitudinal segment 3234 may be curved in three dimensions in order to locate node 3232 in the optimal position over anatomical features. FIG. 34 shows a terminal node that could be located in an optimal location for an iliac bone anchor. The angle of the hole 3233, as shown in FIG. 34, can be angularly offset (e.g., non-parallel) from the primary plane 3247 of plate 3230. The shape and topography of plate 3230, nodes 3232, holes 3233, and longitudinal segment 3234 can be determined using surgical planning and design software. The plate 3230 may comprise multi-planar curves, twists, variable thicknesses, multiple sections of differing density, multiple sections of differing porosity, or multiple sections of differing flexibility/stiffness. Screws can be installed in the holes 3233 to fix the rods 3230 to anatomy.

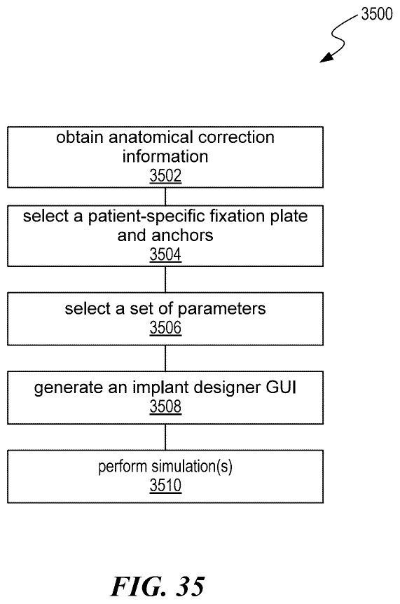

FIG. 35 is a flowchart of a method 3500 for designing drill guides for a patient-specific fixation plate in accordance with embodiments of the present technology. The method 3500 can be used to design a wide range of implant types. For example, the method 3500 can be used or modified to design plates (e.g., as discussed in connection with FIGS. 1-22), implant fixation systems (e.g., as discussed in connection with FIGS. 30-34), cages (e.g., as discussed in connection with FIGS. 28A-29), and other types of implants. Example steps of the method 3500 are discussed below with respect to spinal procedures.

At step 3502, the system obtains a digital model of anatomy of a patient and anatomical correction information. The system can acquire medical imaging data such as CT or MRI scans of the patient's spine and surrounding anatomy. The system then processes this imaging data to generate a detailed three-dimensional digital model of the patient's spinal anatomy. The anatomical correction information, which may include specifics on the desired spinal alignment or deformity correction to be achieved through surgery, can be input by a surgeon or generated through analysis of the patient's anatomy and biomechanical modeling. This digital model and correction information serve as the foundation for designing the patient-specific implant and surgical plan.

At step 3504, the system selects a patient-specific fixation plate (or rods) and anchors configured to cooperate to achieve an anatomical correction based on the anatomical correction information. This selection process can include analyzing the digital model of the patient's anatomy and the desired correction to determine the optimal size, shape, and configuration of the fixation plate. The system considers factors such as the patient's vertebral anatomy, the specific deformity or misalignment to be corrected, and the biomechanical forces that will be acting on the implant. For the anchors, the system calculates ideal trajectories, lengths, and diameters to ensure secure fixation while avoiding critical structures like nerves and blood vessels. The selection also takes into account the surgeon's preferences and any constraints of the surgical approach.

At step 3506, the system selects a set of parameters for designing the drill guides based on the digital model and the anatomical correction information. The system can individually design pairs of the drill guides for respective vertebrae, and generate a virtual model of a fixation plate assembly with the individually designed pairs of the drill guides connected to a fixation plate. The parameters may include the length, diameter, and trajectory of each drill guide, as well as its position and orientation relative to the fixation plate. The system analyzes the patient's vertebral anatomy, the planned screw trajectories, and the desired correction to determine the optimal parameters for each drill guide. It also considers factors such as the surgical approach, potential obstructions, and the need to avoid critical anatomical structures. By individually designing pairs of drill guides for each vertebra, the system ensures that each guide is tailored to the specific anatomy and correction requirements of its target vertebra.

At step 3508, the system generates an implant designer graphical user interface (GUI) for displaying the set of parameters, values for the respective parameters, a planned anatomy of the patient, and a model of the patient-specific implant positioned along the planned anatomy. The GUI can display the set of parameters and their corresponding values that were selected for designing the drill guides. The GUI can present a visual representation of the planned anatomy of the patient, typically in the form of a 3D model derived from the patient's imaging data. Alongside this, the GUI can show a model of the patient-specific implant positioned along the planned anatomy, allowing surgeons and designers to see how the implant will fit and function within the patient's unique spinal structure. This interactive interface enables real-time adjustments and refinements to the implant design, providing a comprehensive tool for optimizing the patient-specific fixation plate assembly.

At step 3510, the system virtually simulates implantation of, for example, a patient-specific fixation plate by drilling holes in a vertebral body of the digital model based on usage of the drill guides and installation of first and second screws in the holes. The system can score the simulation based on anchoring criteria, such as a combined pullout strength for screws in the vertebral body, a screw anti-back-out score, or a screw-plate interaction score.

The simulation process can include using the digital model of the patient's anatomy and the designed drill guides to virtually drill holes in the vertebral bodies. The system accurately replicates the trajectories, depths, and diameters of the holes as they would be created during the actual surgical procedure. This virtual drilling takes into account the specific parameters of each drill guide, ensuring that the simulated holes precisely match those that would be produced in the operating room.

Following the virtual drilling, the system simulates the installation of the first and second screws into the created holes. This simulation considers factors such as screw size, thread design, and insertion torque to accurately represent the screw's interaction with the bone. The system also models the fixation plate's placement and its interface with the screws and vertebral surfaces. Throughout this virtual implantation process, the system continuously analyzes the biomechanical forces at play, including the stress distribution across the implant and surrounding bone structures. This comprehensive simulation allows for a detailed evaluation of the implant's performance and stability before any physical procedure takes place.

The system can simulate intra-intraoperative positioning of the drill guides of the patient-specific fixation plate along the spine. In some cases, the system can simulate the patient-specific fixation plate with the drill guides positioned along the planned anatomy. The system can display the output from the simulation via the implant designer graphical user interface (GUI) (e.g., user interface 2500, 2600, and/or 2700 of FIGS. 25-27). The simulation step 3510 can also include simulations of implanting components (e.g., cages, artificial discs, etc.) before, during, and/or after virtually implanting the plates or rods. In some embodiments, the simulations can be used to determine an implantation plan, including a planned order for the implantation of implants.

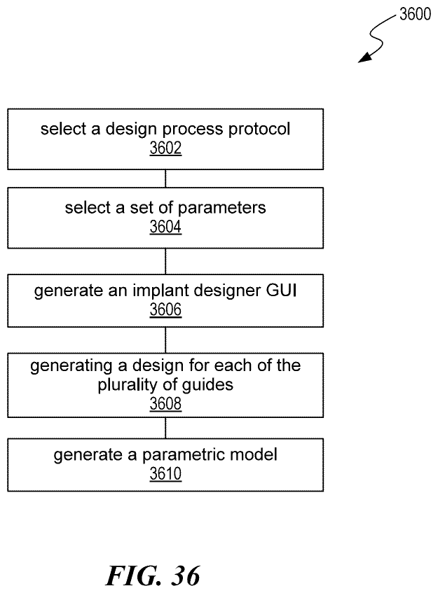

FIG. 36 is a flowchart of a method 3600 for designing drill guides for a patient-specific implant in accordance with embodiments of the present technology. The method 3600 can be used to design a wide range of implant types. For example, the method 3600 can be used or modified to design implant fixation systems (e.g., as discussed in connection with FIGS. 1, 2, 9-14, 30-34), cages (e.g., as discussed in connection with FIGS. 28A-29), and other types of implants. Example steps of the method 3600 are discussed below with respect to spinal procedures.

At step 3602, the system can select a design process protocol for designing a patient-specific fixation plate assembly based on a target correction for a patient. The selection can include analyzing the specific requirements of the patient's case, including the type and severity of the spinal deformity or condition to be corrected. The system may choose from various protocols, such as a user-controlled design process, a machine learning-driven approach, or a hybrid method, depending on the complexity of the case and the surgeon's preferences. Factors considered in this selection may include the patient's anatomy, the desired surgical outcome, the available manufacturing capabilities, and any specific constraints or requirements set by the surgical team.

At step 3604, the system selects a set of parameters for designing each of the plurality of guides based on patient anatomy and the target correction (e.g., spinal realignment) for the patient. The selection of the set of parameters can include analyzing the digital model of the patient's spine and the desired spinal realignment to determine the optimal characteristics for each guide. The system considers factors such as the specific vertebral anatomy, the planned screw trajectories, and the overall correction strategy. Parameters selected may include the length, diameter, and orientation of each guide, as well as its position relative to the fixation plate and surrounding anatomical structures. The system also takes into account surgical approach constraints and the need to avoid critical structures like nerves and blood vessels when determining these parameters.

At step 3606, the system generates an implant designer graphical user interface (GUI) for displaying the set of parameters for the design process protocol, values for the respective parameters, and a planned anatomy of the patient. The design process protocol can include a user-controlled design protocol in which a user inputs the values or a machine learning process protocol in which a machine learning module generates the values. The planned anatomy of the patient can be represented by a virtual model of the anatomy of the patient with the target correction.

At step 3608, the system generates a design for each of the plurality of guides. The guides can be designed such that a fixation plate of the fixation plate assembly is coupled to vertebrae for achieving the anatomical correction after the guides have been separated from the fixation plate and the anchors have been positioned through the fixation plate and along holes in the vertebrae formed using the plurality of guides. This design process can incorporate the patient-specific anatomical data, the planned screw trajectories, and the desired spinal realignment to create guides that will precisely direct the drilling and anchor placement. The system ensures that each guide is optimally positioned and oriented on the fixation plate to achieve the intended anatomical correction once the anchors are in place. It also considers the need for the guides to be easily separable from the fixation plate after use, while maintaining the plate's ability to securely couple to the vertebrae through the drilled holes. In some embodiments, the system generates a model of corrected anatomy and then designs the fixation plate based on the corrected anatomy. For example, the system can design a virtual fixation plate configured to fit planned corrected anatomy. The system can iteratively modify one or more implants and concurrently or sequentially modify designs of other implants.

At step 3610, the system can obtain measurements using a model of the patient anatomy and generate a parametric model of the fixation plate assembly based on the measurements. To generate a parametric model of the fixation plate assembly, the system can utilize the measurements obtained from the patient's anatomical model. It creates a flexible, rule-based representation of the implant that can be easily adjusted based on specific parameters. The parametric model incorporates key design features such as plate dimensions, screw hole positions, and guide orientations, all of which can be dynamically modified. This approach allows for rapid iteration and customization of the implant design to precisely match the patient's anatomy and achieve the desired correction. The system can then virtually position this parametric model along the patient's anatomical model within the user interface, providing a comprehensive visualization of how the customized implant will fit and function in the patient's spine. The system can virtually position, within a user interface, the parametric model of the fixation plate assembly along the model of anatomy of the patient. The system can generate viewable data illustrating the patient-specific implant system virtually positioned in the patient.

The system can modify the parametric model based on a modification to one or more of the values and determine whether the modified parametric model meets at least one design criteria. The system can modify the parametric model by adjusting specific design parameters based on user input or automated optimization algorithms. These modifications can include changes to plate dimensions, screw hole positions, guide orientations, or other critical features. As adjustments are made, the system dynamically updates the model in real-time, allowing for immediate visualization of the changes. The system then evaluates the modified parametric model against predefined design criteria, such as biomechanical performance, manufacturability, and anatomical fit. If the modified model fails to meet these criteria, the system may suggest further adjustments or revert to previous configurations, ensuring that the final design is both patient-specific and clinically viable.

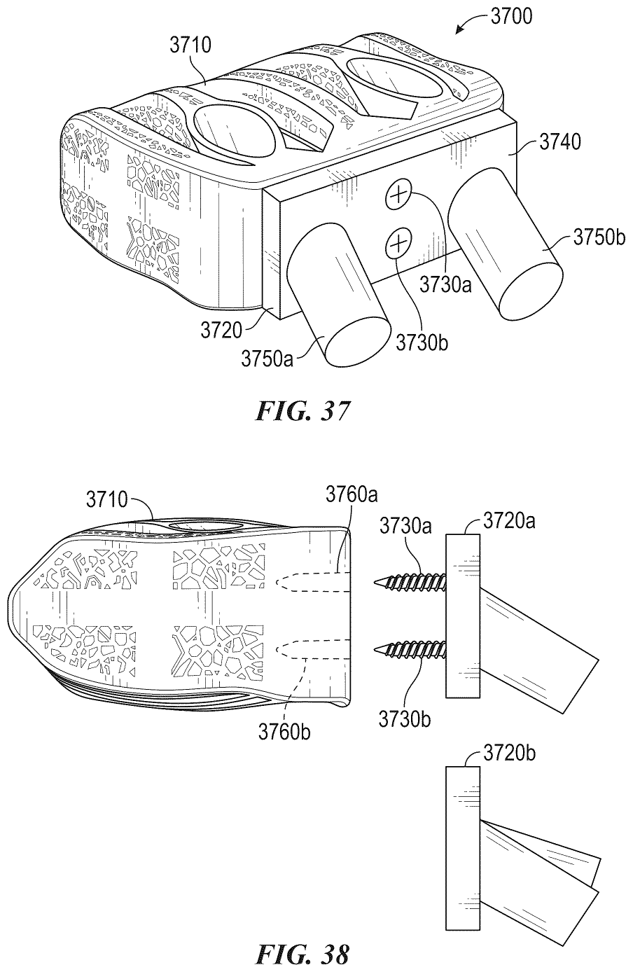

The system can be used to individually or collectively design patient-specific implants, guides, etc. Individual implants, sets of implants, surgical kits, and/or multi-component implant systems can be individually or collectively scored. The scoring can be performed at different times (e.g., during and/or upon completion of steps of methods 3500, 3600). Plans and implant designs can be synchronized such that each is updated when the other is modified. A user and/or a system (e.g., a trained/learning system, machine learning system, artificial intelligence system, etc.) can iteratively modify plans and/or implant designs to develop a desired treatment. The system can design and manufacture multiple components for a surgical kit. Additionally, multiple guides can be designed and manufactured for a single implant. For example, the system can design multiple guides for use during surgery for an implant. Implants, replaceable/removable surgical guides, and surgical kits are discussed in connection with FIGS. 37-39.

FIG. 37 is an isometric view of spinal fusion assembly 3700 in accordance with embodiments of the present technology. The spinal fusion assembly 3700 can include an intervertebral cage 3710 and a temporary guide 3720. The description of the spinal fusion device 2800 of FIGS. 28A-29 applies equally to the device 3700 unless indicated otherwise.

The body or cage 3710 (“cage 3710”) can have one or more features for detachably coupling to the guide 3720. The guide 3720 can include one or more connectors 3730a,b (collectively, “connectors 3730”) that can be used to detachably couple a guide body 3740 to the cage 3710. The guide 3720 can have guide elements 3750a, 3750b usable to deliver bone screws or other components discussed herein. The guide elements 3750 can be configured (e.g., oriented, sized, etc.) based on the surgical procedure to be performed. In some embodiments, the guide body 3740 can be detachably coupled to the cage 3710 with one or more breakaway features. For example, breakaway features discussed in connection with FIGS. 15-19 can be used to temporarily couple the guide 3720 and the cage 3710 together. The cage 3710 can be coupled to any number of guides before, during, and after implantation.

FIG. 38 is a side view of the patient-specific spinal fusion device 3700 and multiple guides 3720. The fasteners 3730a, 3730b can be removed from threaded openings 3760a, 3760b, respectively. The guide 3720a can be replaced with another guide 3720b, which has guide features at different orientations. This allows for customization of implantation during a procedure. In some embodiments, the guide 3720 can be detachably coupled to the implant body 3710 and the guides can be detachably coupled to the guide plate.

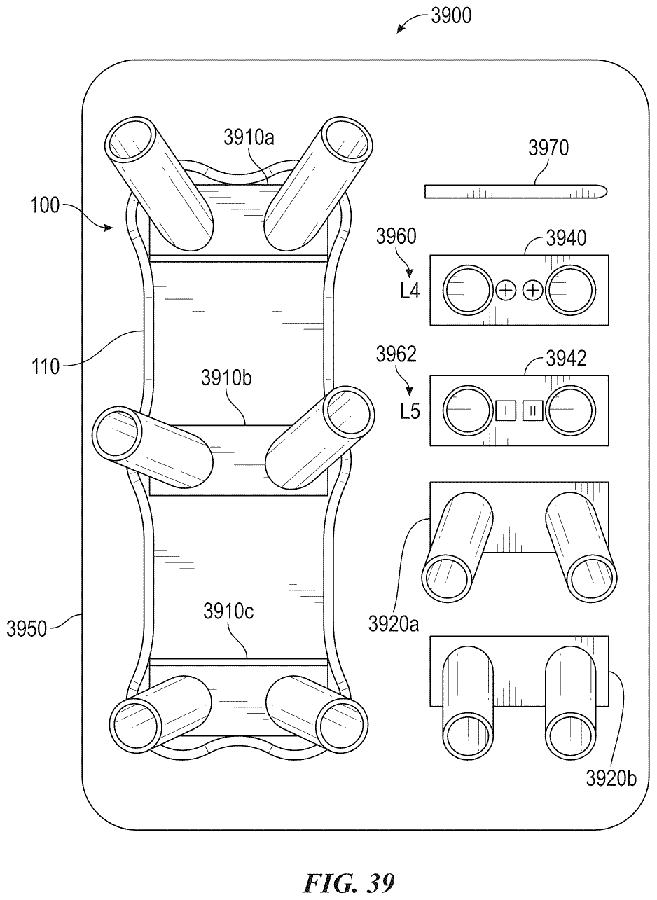

FIG. 39 is a surgical kit 3900 that includes the patient-specific system 100 discussed in connection with FIGS. 1 and 2 in accordance with an embodiment of the technology. The fixation plate assembly 110 can include a plurality of detachable guide assemblies 3910a, 3910b, 3910c. The guide assembly 3910 can be similar to the guide assembly 3720 discussed in connection with FIGS. 37 and 38. Ancillary guides 3920a, 3920b can be used to replace the installed guides 3910.

The kit 3900 can include a number of implants. In the illustrated embodiment, the kit 3900 includes a plurality of spinal fusion devices 3940, 3942. The ancillary plates 3920a, 3920b can be used with the fixation system 100, the implant 3940, and/or the implant 3942. The systems disclosed herein can design and manufacture kits with a set of implants for selection by the surgeon for customizing surgical procedures using patient-specific components, standard components, or the like. The system 2300 of FIG. 23 can synchronize surgical planning with the requirements to deign, manufacture and/or provide patient-specific surgical kits. The surgical kits can include implants, instruments, and components. The illustrated surgical kit 3900 is a patient-specific kit for performing a spinal procedure on a patient and can include the spinal implant 3940 for implantation at a first level and the spinal implant 3942 designed for implantation at a second level. The kit 3900 can include spinal implants (e.g., cages) having different heights (e.g., 3 mm, 4 mm, 5 mm, 6 mm, 7 mm, etc.) to allow a surgeon to position different-sized implants along the spine. For example, if a physician inserts one of the implants 3940 and does not achieve a suitable restored disc height, the physician can implant another implant 3942 until a suitable restored disc height is achieved. The implants can be non-patient-specific implants, patient-specific implants, or combinations thereof. The implants can be patient-specific implants with customized vertebral-contacting surfaces and can be geometrically congruent, include similar vertebral-contacting surfaces, and have different heights.

Surgical kits can include all or some of the implants, components, and features shown in FIGS. 1-39 and can include, for example, kit information, a viewable planned pathology for the patient, and can be configured to receive user input for modifying components of kits, selecting components of kits, etc. The surgical kit 3900 can include a container 3950 with positioning information 3960, 3962 indicating levels for implantation, delivery paths, target anatomical values, etc. For example, the implants 3940 are configured to be planted at a position at L4. The implants 3942 are configured to be planted at a position at L5. The surgical kit 3900 can include one or more instruments 3970. The instruments 3970 can include, for example, inserter instruments, decompression instruments, debulking instruments, rongeurs, or the like. The container 3950 can be a sterilizable tray that can hold instruments, instructions for use, or the like.

The patient-specific implant assembly of FIGS. 37 and 38 can be designed, analyzed, and/or manufactured using the computing system 2300 of FIG. 23. The spinal fusion assembly 3700 can include an implant, such as the intervertebral cage 3710, configured to mate with a plurality of vertebrae and including a plurality of openings. The spinal fusion assembly 3700 can further include a guide assembly, such as the temporary guide 3720, configured to be detachably coupled to the implant and including a guide body 3740 and a plurality of guide elements 3750a, 3750b extending away from a fixation guide body at different orientations. When the guide body 3740 is coupled to the implant, a passageway of one of the plurality of guide elements 3750a, 3750b can be aligned with a corresponding one of the plurality of openings in the implant to allow a drill or an anchor to pass along the passageway, through the corresponding one of the plurality of openings, and into one of the plurality of vertebrae. The treatment planning module 2318 can analyze patient anatomy data to determine user-approved configurations, optimal configurations, etc. for the intervertebral cage 3710 and the guide assembly 3720. The system can design the guide body 3740 to mate with the cage 3710 such that the passageways of the guide elements 3750a, 3750b are aligned with corresponding openings in the cage 3710. The system can determine trajectories for the guide elements 3750a, 3750b based on the patient's vertebral anatomy and the desired anchor placement locations. The breakaway connections or threaded members (e.g., threaded members coupling the guide body 3740 to the cage 3710) can be designed to allow separation after drilling or anchor placement while maintaining structural integrity during use. The system can generate multiple guide assemblies 3720a, 3720b with different guide element orientations for the same cage 3710, allowing surgeons to select the appropriate guide configuration during the surgical procedure.

In some embodiments, the guide body 3740 can be detachably coupled to the cage 3710 by a breakaway connection configured to break apart at a location within a recessed region of the cage 3710. The breakaway connection can include at least one of a frangible feature, a cleave, or a notch, similar to the breakaway connections 600 described in connection with FIGS. 6 and 7. The frangible features can be positioned such that when the guide body 3740 is separated from the cage 3710, the remaining portions of the breakaway connection are recessed below the outer surface of the cage 3710 to avoid irritating adjacent tissue. Alternatively or additionally, at least one member (e.g., pion, threaded member, etc.) can detachably couple the guide body 3740 to the cage 3710. For example, the connectors 3730a, 3730b can be threaded fasteners that engage with the threaded openings 3760a, 3760b to secure the guide body 3740 to the cage 3710 during use and allow removal by unthreading the fasteners after drilling or anchor placement is complete.

The plurality of guide elements 3750a, 3750b can be integrally formed with the guide body 3740, and the guide body 3740 can be configured to lay flat along the cage 3710 when coupled thereto. This configuration allows the guide body 3740 to maintain a low profile relative to the cage 3710 while providing the guide elements 3750a, 3750b at the appropriate orientations for drilling or anchor delivery. The integral formation of the guide elements 3750a, 3750b with the guide body 3740 can provide structural rigidity during use. The number (e.g., one, two, five, etc.) and configuration of the guide elements can be selected based on implantation technique, implantation site, health of anatomy (e.g., bone, soft tissue) of the patient, or the like.

The spinal fusion assembly 3700 can further include a plurality of bone screws (or anchors, spikes, etc.) each configured to be inserted through one of the plurality of openings in the cage 3710 to hold the cage 3710 against one or more of the plurality of vertebrae. Two or more of the plurality of bone screws can have at least one of different diameters or different lengths to accommodate variations in vertebral anatomy and provide optimal fixation at different locations. For example, bone screws positioned in denser cortical bone regions can have different thread characteristics than bone screws positioned in cancellous bone regions.

The cage 3710 can be an intervertebral cage configured for positioning between vertebral endplates, or the implant can be a fixation plate configured for attachment to anterior, lateral, or posterior surfaces of vertebral bodies. The guide assembly 3720 can be adapted for use with either type of implant, with the guide elements 3750a, 3750b oriented to provide appropriate trajectories for the specific implant configuration and surgical approach.

In some embodiments, the guide assembly 3720 is a first guide assembly, and the spinal fusion assembly 3700 further includes a second guide assembly configured to couple to the cage 3710 and include a plurality of guide elements different from the plurality of guide elements of the first guide assembly. As shown in FIG. 38, the guide 3720a can be replaced with the guide 3720b, which has guide features at different orientations. The first guide assembly can be configured to guide drill bits for forming pilot holes, while the second guide assembly can be configured to guide bone screws or anchors into the drilled holes. The different guide assemblies can have guide elements with different diameters, lengths, or angular orientations to accommodate different surgical steps or anchor configurations.

The computing system 2300 can determine one or more design goals for the patient-specific implant assembly and surgical kit based on the patient data set 2308, surgical team data set 2310, and/or facility data set 2312. Design goals can include, for example, reducing the likelihood of subsidence, reducing the likelihood of anchor pullout, reducing implant movement, minimizing recovery time, reducing the length of surgery, improving fusion rates, reducing intraoperative blood loss, minimizing soft tissue trauma, or combinations thereof. The treatment planning module 2318 can analyze the design goals to determine design parameters and constraints for the implant components and guide assemblies. For example, if a design goal is to reduce the likelihood of subsidence, the system can determine parameters such as implant footprint dimensions, endplate contact surface area, surface texture characteristics, and anchor placement locations that distribute loads across the vertebral endplates. If a design goal is to reduce the length of surgery, the system can determine parameters such as the number of guide assemblies, guide element orientations that minimize repositioning, and anchor configurations that reduce the number of insertion steps. The system can prioritize design goals based on user input or patient-specific factors and can generate design parameters that balance competing goals to achieve an optimal overall outcome.

The computing system 2300 can design surgical kits by analyzing the patient's anatomy and surgical plan to determine the components needed for the procedure. The data analysis module 2316 can evaluate the patient data set 2308 to identify the vertebral levels requiring treatment and the types of implants and guides needed at each level. The treatment planning module 2318 can then design patient-specific components including fixation plates with breakaway guide elements, intervertebral cages with detachable guide assemblies, bone screws of varying lengths and diameters, and ancillary guides for replacement during surgery. Conventional implants and bone screws can be installed at locations prone to subsidence, pull out, and implant movement. The system can design the kit 3900 to include the fixation plate assembly 110 with detachable guide assemblies 3910a, 3910b, 3910c, ancillary guides 3920a, 3920b configured to couple to the fixation plate assembly 110 or the spinal fusion devices 3940, 3942, and instruments 3970 for implant delivery. The system can generate positioning information 3960, 3962 indicating the vertebral levels for each component based on the surgical plan. The spinal fusion devices 3940, 3942 can be designed to reduce, limit, or prevent subsidence, pull out, and implant movement.

Conventional surgical techniques present numerous challenges that can compromise surgical outcomes. It can be difficult to determine where to drill tissue using intra-operative imaging with limited viewing capabilities, particularly in anatomically complex regions such as the spine where critical structures including nerves, blood vessels, and the spinal cord are in close proximity to the surgical site. Additionally, freehanded positioning and manipulation of the drill bit can be difficult and produce suboptimal drilling, as surgeons must rely on their experience and visual estimation to achieve proper trajectories and depths. Standard or stock surgical systems may not accommodate the unique biomechanics and anatomical variations of individual patients, leading to compromised implant fit and potentially reduced long-term stability. Furthermore, conventional approaches often require surgeons to mentally translate two-dimensional imaging data into three-dimensional spatial relationships during the procedure, increasing cognitive load and the potential for error. The lack of patient-specific guidance can result in increased surgical time as surgeons make intraoperative adjustments, suboptimal anchor placement that may lead to implant loosening or migration, and in some cases, damage to surrounding anatomical structures.

The system can design components of the surgical kit 3900 such that multiple guides can be used with a single implant (or implant assembly) during the surgical procedure. For example, the system can design a first guide assembly configured to guide drill bits for forming holes in vertebral bodies and a second guide assembly configured to guide bone screws into the drilled holes. The guide assemblies can hold and limit positioning and manipulation of the drill bit to provide optimal drilling. The guide assemblies can be designed with connectors 3730a, 3730b that engage with threaded openings 3760a, 3760b in the implant, allowing the guides to be interchanged during surgery. The system can design the guide elements of different guide assemblies to have different number of guide, dimensions (e.g., guide diameters, lengths, or orientations), and features (e.g., radiopaque) based on the specific surgical step to be performed. The manufacturing system 2324 can manufacture the designed components, and the system can generate instructions for assembling the surgical kit 3900 with the container 3950 organizing the components according to the surgical workflow.

It may be difficult for surgeons to use standard or stock surgical systems due their biomechanics, preferred surgical techniques, etc. This may result in increased surgical time, suboptimal surgical outcomes, etc. The computing system 2300 can design surgical kits based on surgeon preferences, delivery techniques, and insertion paths. The treatment planning module 2318 can receive surgeon preference data from the surgical team data set 2310, including preferred implant sizes, guide configurations, instrument types, and surgical approaches. The system can customize the surgical kit 3900 to include components that align with the surgeon's preferred techniques, such as selecting guide elements with orientations that match the surgeon's typical insertion angles or including instruments that the surgeon routinely uses for implant delivery. The system can also analyze the planned delivery technique to determine the appropriate guide assemblies, selecting breakaway guide elements or detachable guide assemblies based on whether the surgeon prefers to remove guides before or after anchor placement.

The data analysis module 2316 can evaluate historical data from previous procedures performed by the surgeon to identify patterns in component selection and customize future surgical kits to, for example, decrease surgical times, improve surgical outcomes, reduce intraoperative complications, and enhance overall procedural efficiency. These improvements address significant limitations of conventional surgical techniques, which often rely on standardized, off-the-shelf components that may not optimally accommodate the unique anatomical variations and biomechanical requirements of individual patients. Conventional approaches frequently require surgeons to make real-time adjustments during procedures, leading to increased operative duration, potential for suboptimal implant positioning, and elevated risk of complications such as implant loosening, migration, or failure. By analyzing surgeon-specific preferences, historical outcome data, and patient-specific anatomical characteristics, the system can generate customized surgical kits that reduce the cognitive burden on surgeons, minimize the need for intraoperative improvisation, and provide components that are pre-selected and configured to match both the patient's anatomy and the surgeon's preferred techniques. This patient-specific and surgeon-specific approach is expected to result in more precise implant placement, improved biomechanical performance of the implanted devices, reduced operative time, decreased blood loss, faster patient recovery, and improved long-term clinical outcomes compared to conventional surgical techniques that utilize generic components and standardized procedural approaches.

The system can design surgical kit components based on the planned insertion paths and surgical approach for the procedure. The treatment planning module 2318 can analyze the patient's anatomy and the surgical approach (e.g., anterior, posterior, lateral) to determine the optimal insertion paths for implants and anchors. Based on these insertion paths, the system can design guide elements with trajectories that accommodate the surgical corridor and avoid critical anatomical structures. For example, if the surgical plan specifies a lateral approach, the system can design guide assemblies with guide elements oriented to provide appropriate drill bit and anchor trajectories from the lateral access point. The system can also select or design instruments 3970 that are compatible with the planned insertion paths, such as inserter instruments with appropriate lengths and angles for the surgical corridor. The surgical kit 3900 can include positioning information 3960, 3962 that indicates not only the vertebral levels for implantation but also the recommended insertion paths and delivery sequences based on the surgical plan.

Referring to FIGS. 1-39, the virtual models can be multi-dimensional (e.g., two-dimensional or three-dimensional) virtual models and can include, for example, computer-aided design (CAD) data, material data, surface modeling, manufacturing data, or the like. The CAD data can include, for example, solid modeling data (e.g., part files, assembly files, libraries, part/object identifiers, etc.), model geometry, object representations, parametric data, topology data, surface data, assembly data, metadata, etc. The system can generate predicted intra-operative anatomical models, post-operative or corrected anatomical models, surgical plans (e.g., single-stage or multi-stage surgical plans), virtual models of implants, implant design parameters, and instruments using the virtual model of the patient anatomy. Examples of the foregoing are described in U.S. Pat. No. 11,793,577 and U.S. application Ser. No. 16/048, 167, Ser. No. 16/242,877, Ser. No. 16/207,116, Ser. No. 16/352,699, Ser. No. 16/383,215, Ser. No. 16/569,494, Ser. No. 16/699,447, Ser. No. 16/735,222, Ser. No. 16/987,113, Ser. No. 16/990,810, Ser. No. 17/085,564, Ser. No. 17/100,396, Ser. No. 17/342,329, Ser. No. 17/518,524, Ser. No. 17/531,417, Ser. No. 17/835,777, Ser. No. 17/851,487, Ser. No. 17/867,621, Ser. No. 18/373,899, and Ser. No. 17/842,242, each of which is incorporated by reference herein in its entirety.

Virtual models can include a first set of anatomical element models with high-fidelity surface topologies for designing the patient-specific implants. The 3D model can include a second set of anatomical element models for measuring spinal metrics for predicting a surgical outcome associated with implantation of the one or more patient-specific implants. In some cases, one or more of the anatomical element models of the second set have less feature data than all or some of the anatomical element models of the first set. The system can generate the 3D model by positioning, orienting, and/or scaling anatomical element models of the first set of anatomical elements for incorporation into the second set of anatomical elements. The 3D models can be modified to generate new 3D models that represent different stages of treatment plans. The new 3D models can be modified based on acquired patient data, user input, or the like. In some embodiments, one or more 3D models are generated to represent one or more stages of a treatment plan. Implant models with high-fidelity surface topologies can be generated and fit with the anatomical virtual models.

In some embodiments, the system can generate X-ray-fidelity anatomical element models based on one or more standing X-ray images. The system can generate tomographic-fidelity (e.g., polygon counts of number of polygons or triangles used to represent the model's surface; edge and curve smoothness, surface quality (surface roughness, continuity, and the presence of imperfections or artifacts that may affect the model's fidelity), level of geometric detail (inclusion of fine features, intricate structures, and smaller elements), matching to images, etc.) anatomical element models based on a set of tomographic images of the first multi-dimensional image data. The X-ray-fidelity anatomical element models have a resolution below a threshold fidelity and the tomographic-fidelity anatomical element models have a fidelity above the threshold fidelity, resolution above a threshold resolution, etc. The standing X-ray images can be post-operative images captured after the patient has partially or fully completed recovery from a surgical stage.

The system can generate a 3D multi-region spine model (e.g., including a lumbar region, a cervical region, and/or a thoracic region of the patient's spine) of the patient based on the first multi-dimensional image data and a 3D partial spine model (e.g., including a lumbar region model having surface topology data for designing one or more implants) matching the corresponding region of the spine imaged in the second multi-dimensional image data. The system can generate a 3D multi-region simulation model by combining anatomical elements of the 3D partial spine model with the 3D multi-region spine model. The system can replace lower-fidelity anatomical element models (e.g., based on X-ray images) of the 3D multi-fidelity spine model with the corresponding higher-fidelity anatomical elements (e.g., based on CT images or MRI study) of the 3D partial spine model anatomical elements. The system can collect additional multi-dimensional image data in other loading states and generate a replacement virtual model of an anatomical element for placement in the virtual 3D model. Example 3D multi-region spine models are discussed in U.S. application Ser. No. 18/373,899, which is incorporated by reference herein in its entirety. Virtual implants can be added to the 3D multi-region spine models to generate a composite model (e.g., model of implants and anatomy), and parametric parameters between the virtual implants and spine models can be determined. The parametric parameters can be used to dynamically modify multiple parameters of the composite model.

The implant designs can be generated from the virtual model of the implant, or drawn by the user (e.g., drawn via a touch screen). In some embodiments, the system can generate an implant profile based on the viewing perspective and/or implant's current anatomical orientation. In some embodiments, the system can identify one or more image keying features of the implant. Example image keying features can include, for example, opaque markers, edges, or other features of the implant that can be identified using image processing techniques. The system can retrieve image keying feature information from a database containing designs for the implant. For example, a patient-specific implant can have associated virtual models (e.g., three-dimensional virtual model, CAD files, etc.), keying feature files, data for identifying implants, data for determining implant orientations, unique keying features, or the like. The system can match reference image keying features with corresponding features of the implant in the images to determine the position and orientation of the implant in the patient. The keying features can be identified and shown by interfaces (e.g., display 2322), design windows (e.g., windows 2612 and/or 2710), etc.

The system can perform one or more synchronization routines using image data and non-image data to command the image system (e.g., camera system, robotic C-Arm imaging system, X-ray system) and/or provide instructions for obtaining additional images. For example, synchronization routines can include matching landmarks (e.g., keying features) to synchronize or nearly synchronize images (e.g., images taken for performing checks) with one or more virtual models, pre-operative plans, intra-operative plans, or the like. Additionally or alternatively, the system can retrieve and manipulate components of the virtual 3D model based on the captured images. For example, the components of a virtual 3D model can be manipulated to be aligned with the radiograph taken by the cameras, X-ray, C-Arm, or the like. The virtual 3D model (or components thereof) can be manipulated (e.g., by zooming, stretching, cropping, and/or rotating the virtual 3D model) to align the virtual 3D model with radiograph. The virtual 3D model can include an anatomical model representing anatomy of a patient, implant model, instrument model, or the like. In some embodiments, the alignment can be performed using one or more best fit routines using, for example, one or more edge detection routines, segmentation routines, filtering routines, image recognition routines, or combinations thereof. The system can confirm placement of an implant by confirming that the implant in the intra-operative image (e.g., the radiograph) is in the same placement as the implant in the pre-operative surgical plan. The placement can be scored based on differences between the pre-operative and intra-operative images. The scoring routine can determine the distance between a target position window and the actual position of the implant. If the actual position is within the target position window, the system can indicate the implant is located at the target location. The target position window can be determined using ML models, user input, or the like. In some embodiments, the system can confirm that the implant is positioned at a target location based on identified portion(s) of the implant contacting targeted anatomical feature(s).

In some embodiments, the system can perform real-time checks using one or more captured images (e.g., sequentially captured images obtained using a C-Arm machine) within an augmented reality (AR) application. The system can use a camera feature within the AR application to view intra-operative radiograph images on a user interface. The camera feature of the AR application does not require a camera on the user interface to take the intra-operative image; rather, it displays the intra-operative radiograph images on the user interface. A radiograph image can be taken prior to implantation of the implant, after removal of breakaway features, prior to implantation of screws, after implantation of screws, etc.

The system can perform any number of implant designs and position checks to confirm that the implant design and/or location is acceptable. The position checks can be non-invasive image-guided checks for intra-operatively analyzing the current location of the implant based on obtained images of the patient. The system can identify the implant in the images and then synchronize implant data in the surgical plan with the patient images. For example, the system can synchronize a virtual anatomical model of the surgical plan with radiograph images and then compare the position of the physical implant to a target or acceptable implant position. This process can be repeated until the implant is positioned at an acceptable location in the patient based on the comparison. During a surgical procedure, images can be repeatedly taken to evaluate delivery of the implant.

### CONCLUSION

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2017, titled “SYSTEMS AND METHODS FOR ASSISTING AND AUGMENTING SURGICAL PROCEDURES”;
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY”;
U.S. application Ser. No. 16/207,116, filed on Dec. 1, 2018, titled “SYSTEMS AND METHODS FOR MULTI-PLANAR ORTHOPEDIC ALIGNMENT”;
U.S. application Ser. No. 16/352,699, filed on Mar. 13, 2019, titled “SYSTEMS AND METHODS FOR ORTHOPEDIC IMPLANT FIXATION”;
U.S. application Ser. No. 16/383,215, filed on Apr. 12, 2019, titled “SYSTEMS AND METHODS FOR ORTHOPEDIC IMPLANT FIXATION”;
U.S. application Ser. No. 16/569,494, filed on Sep. 12, 2019, titled “SYSTEMS AND METHODS FOR ORTHOPEDIC IMPLANTS”;
U.S. Application No. 62/773,127, filed on Nov. 29, 2018, titled “SYSTEMS AND METHODS FOR ORTHOPEDIC IMPLANTS”;
U.S. Application No. 62/928,909, filed on Oct. 31, 2019, titled “SYSTEMS AND METHODS FOR DESIGNING ORTHOPEDIC IMPLANTS BASED ON TISSUE CHARACTERISTICS”;
U.S. application Ser. No. 16/735,222 (now U.S. Patent No. 10,902,944), filed Jan. 6, 2020, titled “PATIENT-SPECIFIC MEDICAL PROCEDURES AND DEVICES, AND ASSOCIATED SYSTEMS AND METHODS”;
U.S. Application No. 16/987, 113, filed Aug. 6, 2020, titled “PATIENT-SPECIFIC ARTIFICIAL DISCS, IMPLANTS AND ASSOCIATED SYSTEMS AND METHODS”;
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS”;
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS”;
U.S. application Ser. No. 17/100,396, filed Nov. 20, 2020, titled “PATIENT-SPECIFIC VERTEBRAL IMPLANTS WITH POSITIONING FEATURES”;
U.S. application Ser. No. 17/124,822, filed Dec. 17, 2020, titled “PATIENT-SPECIFIC MEDICAL PROCEDURES AND DEVICES, AND ASSOCIATED SYSTEMS AND METHODS”;
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS”;
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES”;
International Application No. PCT/US 2021/012065, filed Jan. 4, 2021, titled “PATIENT-SPECIFIC MEDICAL PROCEDURES AND DEVICES, AND ASSOCIATED SYSTEMS AND METHODS”;
International Patent Application No. PCT/US 22/48729, filed Nov. 2, 2022, titled “PATIENT-SPECIFIC ARTHROPLASTY DEVICES AND ASSOCIATED SYSTEMS AND METHODS”;
U.S. application Ser. No. 18/113,573, filed Feb. 23, 2023, titled “PATIENT-SPECIFIC IMPLANT DESIGN AND MANUFACTURING SYSTEM WITH A DIGITAL FILING CABINET MANAGER”;
U.S. application Ser. No. 17/878,633, filed Aug. 1, 2022, titled “NON-FUNGIBLE TOKEN SYSTEMS AND METHODS FOR STORING AND ACCESSING HEALTHCARE DATA”;
U.S. Pat. No. 11,806,241, issued Nov. 7, 2023, titled “SYSTEM FOR MANUFACTURING AND PRE-OPERATIVE INSPECTING OF PATIENT-SPECIFIC IMPLANTS”;
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME”;
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS”;
U.S. Pat. No. 11,793,577, issued Oct. 24, 2023, titled “TECHNIQUES TO MAP THREE-DIMENSIONAL HUMAN ANATOMY DATA TO TWO-DIMENSIONAL HUMAN ANATOMY DATA”;
International Patent Application No. PCT/US 22/48729, filed Nov. 2, 2022, titled “PATIENT-SPECIFIC ARTHROPLASTY DEVICES AND ASSOCIATED SYSTEMS AND METHODS”;
U.S. application Ser. No. 18/113,573, filed Feb. 23, 2023, titled “PATIENT-SPECIFIC IMPLANT DESIGN AND MANUFACTURING SYSTEM WITH A DIGITAL FILING CABINET MANAGER”;
U.S. application Ser. No. 17/878,633, filed Aug. 1, 2022, titled “NON-FUNGIBLE TOKEN SYSTEMS AND METHODS FOR STORING AND ACCESSING HEALTHCARE DATA”;
U.S. Pat. No. 11,806,241, issued Nov. 7, 2023, titled “SYSTEM FOR MANUFACTURING AND PRE-OPERATIVE INSPECTING OF PATIENT-SPECIFIC IMPLANTS”;
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME”;
U.S. application Ser. No. 18/384,762, filed Oct. 27, 2023, titled “SYSTEMS AND METHODS FOR SELECTING, REVIEWING, MODIFYING, AND/OR APPROVING SURGICAL PLANS”;
U.S. application Ser. No. 18/408,452, filed Jan. 9, 2024, titled “SYSTEM FOR MODELING PATIENT SPINAL CHANGES”;
U.S. application Ser. No. 18/415,577, filed Jan. 17, 2024, titled “PATIENT-SPECIFIC IMPLANT DESIGN AND MANUFACTURING SYSTEM WITH A SURGICAL IMPLANT POSITIONING MANAGER”;
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS”;
U.S. Pat. No. 11,793,577, issued Oct. 24, 2023, titled “TECHNIQUES TO MAP THREE-DIMENSIONAL HUMAN ANATOMY DATA TO TWO-DIMENSIONAL HUMAN ANATOMY DATA”;
PCT Application No. PCT/US 24/10202, filed Jan. 3, 2024, titled “PATIENT-SPECIFIC SPINAL FUSION DEVICES AND ASSOCIATED SYSTEMS AND METHODS”;
U.S. application Ser. No. 18/892,151, filed Sep. 20, 2024, titled “ROTATABLE AND SURGICAL APPROACH-SPECIFIC INTERVERTEBRAL IMPLANTS FOR FUSION TECHNIQUES”; and
U.S. Application No. 63/717,251, filed Nov. 6, 2024, titled “INTRA-OPERATIVELY MODIFIED IMPLANTS FOR SURGICAL PROCEDURES.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

1. A patient-specific implant, comprising:
a fixation plate configured to mate with a plurality of vertebrae and including a plurality of recessed regions each having an opening; and
a plurality of guide elements extending away from the fixation plate at different orientations and each being connected to the fixation plate by a respective breakaway connection such that a passageway of a guide element is aligned with a corresponding opening in the fixation plate to allow a drill or an anchor to pass along the passageway, through the corresponding opening, and into one of the plurality of vertebrae, and wherein the respective breakaway connection is configured to break apart at a location within a recessed region.

2. The patient-specific implant of claim 1, wherein the fixation plate has a patient-specific geometry contoured to mate with each of the plurality of vertebrae, and wherein each of the plurality of guide elements has a respective passageway defining a trajectory for forming a drill hole or positioning the anchor in the plurality of vertebrae.

3. The patient-specific implant of claim 1, wherein the respective breakaway connection includes at least one of a frangible feature, a cleave, or a notch.

4. The patient-specific implant of claim 1, wherein the location is spaced apart from an opening to a recess, further comprising a bone screw including a head configured to fit in the recess and cover a portion of the respective breakaway connection connected to the fixation plate after removing the guide element.

5. The patient-specific implant of claim 1, wherein the plurality of guide elements is integrally formed with the fixation plate.

6. The patient-specific implant of claim 1, further comprising a plurality of bone screws each configured to be inserted through one of a plurality of openings to hold the fixation plate against one of the plurality of vertebrae.

7. The patient-specific implant of claim 6, wherein two or more of the plurality of bone screws have at least one of different diameters or different lengths.

8. The patient-specific implant of claim 1, further comprising a connection mechanism configured to couple the fixation plate to an intervertebral spacer configured to be positioned between two vertebral bodies of the plurality of vertebrae.

9. A patient-specific implant assembly, comprising:
an implant configured to mate with a plurality of vertebrae and including a plurality of openings; and
a guide assembly configured to be detachably coupled to the implant and including a guide body and a plurality of guide elements extending away from a fixation guide body at different orientations, wherein when the guide body is coupled to the implant, a passageway of one of the plurality of guide elements is aligned with a corresponding one of the plurality of openings in the implant to allow a drill or an anchor to pass along the passageway, through the corresponding one of the plurality of openings, and into one of the plurality of vertebrae.

10. The patient-specific implant assembly of claim 9, further comprising a breakaway connection detachably coupling the guide body to the implant, and wherein the breakaway connection is configured to break apart at a location within a recessed region of the implant.

11. The patient-specific implant assembly of claim 10, wherein the breakaway connection includes at least one of a frangible feature, a cleave, or a notch.

12. The patient-specific implant assembly of claim 9, further comprising at least one threaded member detachably coupling the guide body to the implant.

13. The patient-specific implant assembly of claim 9, wherein the plurality of guide elements is integrally formed with the guide body, and wherein the guide body is configured to lay flat along the implant.

14. The patient-specific implant assembly of claim 9, wherein the guide body includes at least one positioning protrusion.

15. The patient-specific implant assembly of claim 9, further comprising a plurality of bone screws each configured to be inserted through one of the plurality of openings to hold the implant against one of the plurality of vertebrae.

16. The patient-specific implant assembly of claim 15, wherein two or more of the plurality of bone screws have at least one of different diameters or different lengths.

17. The patient-specific implant assembly of claim 9, wherein the implant is an intervertebral cage or a fixation plate.

18. The patient-specific implant assembly of claim 9, wherein the guide assembly is a first guide assembly, further comprising a second guide assembly configured to couple to the implant and include a plurality of guide elements different from the plurality of guide elements of the first guide assembly.

19. A method comprising:
obtaining a digital model of anatomy of a patient and anatomical correction information;
designing a patient-specific fixation plate and anchors configured to cooperate to achieve an anatomical correction based on the anatomical correction information; and
designing a plurality of drill or anchor guides for the patient-specific fixation plate by
selecting a set of parameters for designing the plurality of drill or anchor guides based on the digital model and the anatomical correction information, and
generating an implant designer graphical user interface (GUI) for displaying the set of parameters, respective values for the set of parameters, a planned anatomy of the patient, and a model of the patient-specific fixation plate positioned along the planned anatomy, wherein the model of the patient-specific fixation plate represents the respective values.

20. The method of claim 19, wherein the plurality of drill or anchor guides includes a first drill guide and a second drill guide, the method further including:
determining positional information for the first drill guide and the second drill guide for guiding one or more drill bits into a vertebral body of the digital model;
determining a first trajectory for the first drill guide and a second trajectory for the second drill guide; and
generating a virtual model of the first drill guide and the second drill guide connected to the patient-specific fixation plate.

21.-44. (canceled)
